SEQUENCE LISTING (2 sequences)

SEQ ID NO: 1 - protein, 36 aa, Artificial Sequence
  Description of Artificial Sequence Synthetic polypeptide
YTSLIHSLIEESQNQQEKNEQELLELDKWASLWNWF

SEQ ID NO: 2 - DNA, 21 bp, Artificial Sequence
  Description of Artificial Sequence Synthetic oligonucleotide
gcgtttgctcttcttcttgcg

(19) United States
(12) Patent Application Publication
(10) Pub. No.: US 2018/0221406 A1
(43) Pub. Date: Aug. 9, 2018

(54) THERAPEUTIC COMBINATIONS OF ANTIVIRAL AND ANTI-INFLAMMATORY THERAPIES

(71) Applicant: Foresight Biotherapeutics, Inc., New York, NY (US)

(72) Inventors: Jason STEIN, New York, NY (US); Michael WEISER, New York, NY (US)

(73) Assignee: Foresight Biotherapeutics, Inc., New York, NY (US)

(21) Appl. No.: 15/570,051

(22) PCT Filed: Apr. 28, 2016
(86) PCT No.: PCT/US16/29677

§ 371 Date: Apr. 9, 2018

Related U.S. Application Data
(60) Provisional application No. 62/154,478, filed on Apr. 29, 2015.

(51) Int. Cl. A61K 33/18; A61K 9/00; A61P 31/20

(52) U.S. Cl. CPC A61K 33/18; A61K 9/0048; A61K 31/573; A61K 2300/00; A61P 31/20

(57) ABSTRACT

In some embodiments, the invention includes therapeutic combinations of antiviral active pharmaceutical ingredients and anti-inflammatory active pharmaceutical ingredients, including steroids, and methods of using of the therapeutic compositions in the treatment of viral infections.

### FIELD OF THE INVENTION

Therapeutic combinations of antiviral active pharmaceutical ingredients and anti-inflammatory agents and uses thereof are disclosed herein.

### BACKGROUND OF THE INVENTION

Anti-viral active pharmaceutical ingredients are commonly contraindicated with steroids. Steroids inhibit immune response to reduce inflammation and tissue damage, but with the undesirable effect of limiting the body's intrinsic ability to fight infections. Use of anti-inflammatory drugs, including steroids, is thought to worsen the course of a viral infection. For example, it is believed that treatment of ocular infections with herpes simplex virus, particularly infections with live virus, should not be treated with steroids. G. Weiner, “Demystifying the ocular herpes simplex virus: Basic principles and common misconceptions,” Eyenet, January 2013, pp. 40-46 (The American Academy of Opthalmology). Steroids such as dexamethasone have also been found to be ineffective in bronchiolitis caused by respiratory syncytial virus (RSV). H. M. Corneli, et al., N. Engl. J. Med. 2007, 357, 331-39. Similarly, a study of prednisolone in uncomplicated acute rhinosinusitis principally of viral origin also indicated that steroid therapy was ineffective. R. P. Venekamp, et al., Can. Med. Assoc. J. 2012, 184, E751-57. Steroids are believed to promote viral shedding and enhance the spread of viral infections. For example, the commonly-used steroid dexamethasone is known to cause increased viral proliferation and viral spreading.

There is an urgent need to provide faster and more effective treatment options for viral infections. Current treatments do not kill virus with sufficient speed to prevent interpatient and intrapatient spreading of infection. For example, acute conjunctivitis affects approximately 6 million people each year in the United States, and the vast majority of cases are adenoviral in origin. No approved treatment exists for adenovirus infection of the eye. As a result, acute conjunctivitis is commonly misdiagnosed as bacterial and can lead to overuse of anti-bacterial eye drops and increased bacterial resistance. The present invention provides methods based on a combination of an antiviral active pharmaceutical ingredient with an anti-inflammatory active pharmaceutical ingredient such as a steroid, which unexpectedly can lead to improved efficacy and faster cure rates for a number of different types of viral infections, including opthalmological viral infections.

### SUMMARY OF THE INVENTION

In an embodiment, the invention provides a pharmaceutical composition comprising an antiviral active pharmaceutical ingredient and an anti-inflammatory active pharmaceutical ingredient, such as a steroid. In an embodiment, the invention provides a pharmaceutical composition comprising an antiviral active pharmaceutical ingredient and a steroid in combination for use in the treatment of viral diseases. In an embodiment, the invention provides method of treating viral diseases comprising the step of administering an antiviral active pharmaceutical ingredient and an anti-inflammatory active pharmaceutical ingredient, such as a steroid, in combination.

In an embodiment, the invention provides a kit comprising a composition comprising an antiviral active pharmaceutical ingredient and an anti-inflammatory active pharmaceutical ingredient, such as a steroid, for use in the treatment of viral infections. The compositions are typically both pharmaceutical compositions. The kit is for use in co-administration of the antiviral active pharmaceutical ingredient and the anti-inflammatory active pharmaceutical ingredient, including a steroid, either simultaneously or separately.

### BRIEF DESCRIPTION OF THE DRAWINGS

The foregoing summary, as well as the following detailed description of the invention, will be better understood when read in conjunction with the appended drawings.

FIG. 1 illustrates the results of viral eradication in an adenoviral rabbit model for dexamethasone 0.1% and povidone-iodine 0.6% (“0.1% dex/0.6% PVP-I”), shown in comparison to TOBRADEX (0.3% tobramycin and 0.1% dexamethasone), 5% cidofovir, and placebo.

### DETAILED DESCRIPTION OF THE INVENTION

Unless defined otherwise, all technical and scientific terms used herein have the same meaning as is commonly understood by one of skill in the art to which this invention belongs.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The terms “co-administration” and “administered in combination with” as used herein, encompass administration of two or more active pharmaceutical ingredients to a subject (such as a human or a mammal), so that both agents and/or their metabolites are present in the subject at the same time. Active pharmaceutical ingredients are also referred to as agents, active ingredients, or drugs. Co-administration includes simultaneous administration in separate compositions (also referred to as concurrent administration), administration at different times in separate compositions, or administration in a composition in which both agents are present. Simultaneous administration in separate compositions and administration in a composition in which both agents are present are preferred.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Supplementary active pharmaceutical ingredients, such as other drugs, can also be incorporated into the described compositions and methods.

“Prodrug” is intended to describe a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active compound.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 10%, preferably from 0% to 10%, more preferably from 0% to 5% of the stated number or numerical range. The term “comprising” (and related terms such as “comprise” or “comprises” or “having” or “including”) encompasses those embodiments such as, for example, an embodiment of any composition of matter, method or process that “consist of” or “consist essentially of” the described features.

“Alkyl” refers to a straight or branched hydrocarbon chain radical consisting solely of carbon and hydrogen atoms, containing no unsaturation, having from one to ten carbon atoms (i.e., C₁-C₁₀alkyl and (C₁-C₁₀)alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, heptyl, octyl, nonyl and decyl. The alkyl moiety may be attached to the rest of the molecule by a single bond, such as for example, methyl (Me), ethyl (Et), n-propyl (Pr), 1-methylethyl (iso-propyl), n-butyl, n-pentyl, 1,1-dimethylethyl (t-butyl) and 3-methylhexyl. Unless stated otherwise specifically in the specification, an alkyl group is optionally substituted by one or more of substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, unsubstituted alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., C₂-C₁₀ alkenyl and (C₂-C₁₀)alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an (alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e. C₂-C₁₀ alkynyl and (C₂-C₁₀)alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. C₂-C₁₀ cycloalkyl and (C₂-C₁₀)cycloalkyl). Whenever it appears herein, a numerical range such as “3 to 10” refers to each integer in the given range—e.g., “3 to 10 carbon atoms” means that the cycloalkyl group may consist of 3 carbon atoms and greater, up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein “R” is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound of Formula (I), thereby forming a prodrug. The procedures and specific groups to make such amides, including the use of protecting groups, are known to those of skill in the art and can readily be found in seminal sources such as Greene and Wuts, Protective Groups in Organic Synthesis, 3^{rd} Ed., John Wiley & Sons, New York, N.Y., 1999.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters, including the use of protecting groups, are known to those of skill in the art and can readily be found in seminal sources such as Greene and Wuts, Protective Groups in Organic Synthesis, 3^{rd} Ed., John Wiley & Sons, New York, N.Y., 1999. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-d]pyridazinyl, dibenzofuranyl, dibenzothiophenyl, furanyl, furazanyl, furanonyl, furo[3,2-d]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-d]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-d]pyridinyl, and thiophenyl (i.e. thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)- and 20% (R)-, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

The terms “enantiomerically enriched,” “enantiomerically pure” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, such as at least 80% by weight. In some embodiments, the enrichment can be significantly greater than 80% by weight, providing a “substantially enantiomerically enriched,” “substantially enantiomerically pure” or a “substantially non-racemic” preparation, which refers to preparations of compositions which have at least 85% by weight of one enantiomer relative to the other enantiomer, such as at least 90% by weight, and such as at least 95% by weight. The terms “diastereomerically enriched” and “diastereomerically pure,” as used herein, refer to compositions in which the percent by weight of one diastereomer is greater than the amount of that one diastereomer in a control mixture of diastereomers. In some embodiments, the enrichment can be significantly greater than 80% by weight, providing a “substantially diastereomerically enriched” or “substantially diastereomerically pure” preparation, which refers to preparations of compositions which have at least 85% by weight of one diastereomer relative to other diastereomers, such as at least 90% by weight, and such as at least 95% by weight.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York, 1981; and Eliel, Stereochemistry of Carbon Compounds, McGraw-Hill, N Y, 1962.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed after the selective reaction is complete. A variety of protecting groups are disclosed, for example, in T. H. Greene and P. G. M. Wuts, Protective Groups in Organic Synthesis, Third Edition, John Wiley & Sons, New York, 1999.

“Solvate” refers to a compound in physical association with one or more molecules of a pharmaceutically acceptable solvent.

“Substituted” means that the referenced group may have attached one or more additional moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “substituted” also means that one or more hydrogens on the designated atom/atoms is/are replaced with a selection from the indicated group, provided that the designated atom's normal valency under the existing circumstances is not exceeded, and that the substitution results in a stable compound. Combinations of substituents and/or variables are permissible if such combinations result in stable compounds. “Stable compound” or “stable structure” is defined as a compound or structure that is sufficiently robust to survive isolation to a useful degree of purity from a reaction mixture, and formulation into an efficacious therapeutic agent. The terms “optionally substituted” and “may optionally be substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Co-Administration of Compounds

In an embodiment, the invention provides a pharmaceutical composition comprising an antiviral active pharmaceutical ingredient and a steroid. In an embodiment, the invention provides a pharmaceutical composition comprising an antiviral active pharmaceutical ingredient and a steroid in combination for use in the treatment of viral diseases. In an embodiment, the invention provides method of treating viral diseases comprising the step of administering an antiviral active pharmaceutical ingredient and a steroid in combination.

In an embodiment, the invention provides a kit comprising a composition comprising an antiviral active pharmaceutical ingredient and a steroid for use in the treatment of viral infections. The compositions are typically both pharmaceutical compositions. The kit is for use in co-administration of the antiviral active pharmaceutical ingredient and the steroid, either simultaneously or separately.

The combination may be administered by any route known in the art. In an embodiment, the combination of the antiviral active pharmaceutical ingredient and steroid is administered by oral, inhaled, intravenous, intramuscular, intraperitoneal, subcutaneous or transdermal means. In one embodiment, the administration is by injection.

In an embodiment, the antiviral active pharmaceutical ingredient and steroid each are in the form of a pharmaceutically acceptable salt.

In an exemplary embodiment, the subject is a mammal, such as a human.

### Antiviral Active Pharmaceutical Ingredients

In an embodiment, the antiviral active pharmaceutical ingredient is povidone-iodine (PVP-I). PVP-I is a stable complex of polyvinylpyrrolidone (PVP, povidone) and about 9-12% w/w elemental iodine. PVP-I is also known as BETADINE, WOKADINE, and PYODINE.

In an embodiment, an antiviral active pharmaceutical ingredient is an antiretroviral drug used against human immunodeficiency virus (HIV). In an embodiment, the antiretroviral drug used against HIV is an entry/fusion inhibitor, such as maraviroc or enfuvirtide.

In an embodiment, the antiviral active pharmaceutical ingredient is [(1S,4R)-4-[2-amino-6-(cyclopropylamino)purin-9-yl]cyclopent-2-en-1-yl]methanol. In a preferred embodiment, the antiviral active pharmaceutical ingredient is abacavir. In a preferred embodiment, the antiviral active pharmaceutical ingredient has the chemical structure shown in Formula 1:

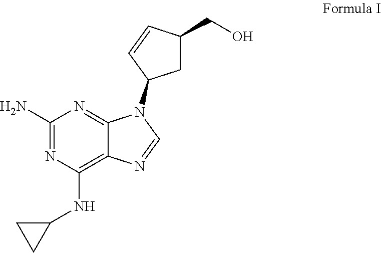 [SMILES: Nc1nc(NC2CC2)c2ncn([C@H]3C=C[C@@H](CO)C3)c2n1]

or a pharmaceutically acceptable salt, solvate, hydrate, cocrystal, or prodrug thereof. The preparation of this compound is described in U.S. Pat. Nos. 5,089,500, 6,294,540 and 6,641,843, the disclosures of which are incorporated by reference herein. In an embodiment, the antiviral active pharmaceutical ingredient is selected from the compounds described in U.S. Pat. Nos. 5,089,500, 6,294,540 and 6,641,843, the disclosures of which are incorporated by reference herein.

In an embodiment, the antiviral active pharmaceutical ingredient is 2-amino-9-(2-hydroxyethoxymethyl)-3H-purin-6-one. In a preferred embodiment, the antiviral active pharmaceutical ingredient is aciclovir or acyclovir. In a preferred embodiment, the antiviral active pharmaceutical ingredient has the chemical structure shown in Formula 2:

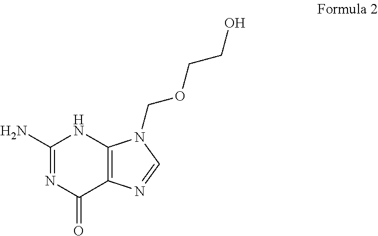 [SMILES: Nc1nc(=O)c2ncn(COCCO)c2[nH]1]

or a pharmaceutically acceptable salt, solvate, hydrate, cocrystal, or prodrug thereof. The preparation of this compound is described in U.S. Pat. Nos. 4,146,715; 8,791,127; 8,592,434; and 8,747,896, the disclosures of which are incorporated by reference herein. In an embodiment, the antiviral active pharmaceutical ingredient is selected from the compounds described in U.S. Pat. Nos. 4,146,715; 8,791,127; 8,592,434; and 8,747,896, the disclosures of which are incorporated by reference herein.

In an embodiment, the antiviral active pharmaceutical ingredient is 2-(6-aminopurin-9-yl)ethoxymethylphosphonic acid. In a preferred embodiment, the antiviral active pharmaceutical ingredient is adefovir. In a preferred embodiment, the antiviral active pharmaceutical ingredient has the chemical structure shown in Formula 3:

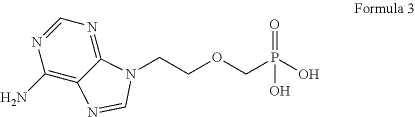 [SMILES: Nc1ncnc2c1ncn2CCOCP(=O)(O)O]

or a pharmaceutically acceptable salt, solvate, hydrate, cocrystal, or prodrug thereof. The preparation of this compound is described in U.S. Pat. Nos. 5,663,159 and 6,451,340, the disclosures of which are incorporated by reference herein. In an embodiment, the antiviral active pharmaceutical ingredient is selected from the compounds described in U.S. Pat. Nos. 5,663,159 and 6,451,340, the disclosures of which are incorporated by reference herein.

In an embodiment, the antiviral active pharmaceutical ingredient is [2-(6-aminopurin-9-yl)ethoxymethyl-(2,2-dimethylpropanoyloxymethoxy)phosphoryl]oxymethyl 2,2-dimethylpropanoate. In a preferred embodiment, the antiviral active pharmaceutical ingredient is adefovir dipivoxil. In a preferred embodiment, the antiviral active pharmaceutical ingredient has the chemical structure shown in Formula 4:

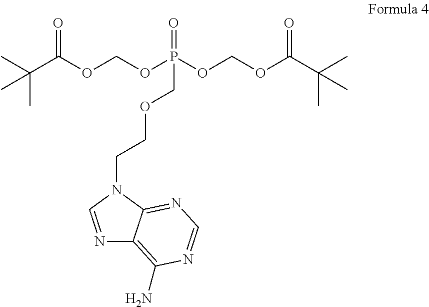 [SMILES: CC(C)(C)C(=O)OCOP(=O)(COCCn1cnc2c(N)ncnc21)OCOC(=O)C(C)(C)C]

or a pharmaceutically acceptable salt, solvate, hydrate, cocrystal, or prodrug thereof. The preparation of this compound is described in U.S. Pat. Nos. 5,663,159 and 6,451,340, the disclosures of which are incorporated by reference herein. In an embodiment, the antiviral active pharmaceutical ingredient is selected from the compounds described in U.S. Pat. Nos. 5,663,159 and 6,451,340, the disclosures of which are incorporated by reference herein.

In an embodiment, the antiviral active pharmaceutical ingredient is adamantan-1-amine. In a preferred embodiment, the antiviral active pharmaceutical ingredient is amantadine. In a preferred embodiment, the antiviral active pharmaceutical ingredient has the chemical structure shown in Formula 5:

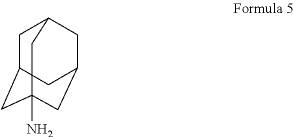 [SMILES: NC12CC3CC(CC(C3)C1)C2]

or a pharmaceutically acceptable salt, solvate, hydrate, cocrystal, or prodrug thereof. The preparation of this compound is described, for example, in U.S. Pat. No. 3,152,180.

In an embodiment, the antiviral active pharmaceutical ingredient is [(3S)-oxolan-3-yl] N-[(2S,3R)-4-[(4-aminophenyl)sulfonyl-(2-methylpropyl)amino]-3-hydroxy-1-phenylbutan-2-yl]carbamate. In a preferred embodiment, the antiviral active pharmaceutical ingredient is amprenavir. In a preferred embodiment, the antiviral active pharmaceutical ingredient has the chemical structure shown in Formula 6:

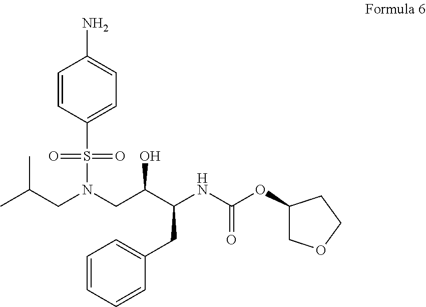 [SMILES: CC(C)CN(C[C@@H](O)[C@H](Cc1ccccc1)NC(=O)O[C@H]1CCOC1)S(=O)(=O)c1ccc(N)cc1]

or a pharmaceutically acceptable salt, solvate, hydrate, cocrystal, or prodrug thereof. The preparation of this compound is described in U.S. Pat. Nos. 5,585,397 and 6,730,679, the disclosures of which are incorporated by reference herein. In an embodiment, the antiviral active pharmaceutical ingredient is selected from the compounds described in U.S. Pat. Nos. 5,585,397 and 6,730,679, the disclosures of which are incorporated by reference herein.

In an embodiment, the antiviral active pharmaceutical ingredient is methyl N-[(2S)-1-[2-[(2S,3S)-2-hydroxy-3-[[(2S)-2-(methoxycarbonylamino)-3,3-dimethylbutanoyl]amino]-4-phenylbutyl]-2-[(4-pyridin-2-ylphenyl)methyl]hydrazinyl]-3,3-dimethyl-1-oxobutan-2-yl]carbamate. In a preferred embodiment, the antiviral active pharmaceutical ingredient is atazanavir. In a preferred embodiment, the antiviral active pharmaceutical ingredient has the chemical structure shown in Formula 7:

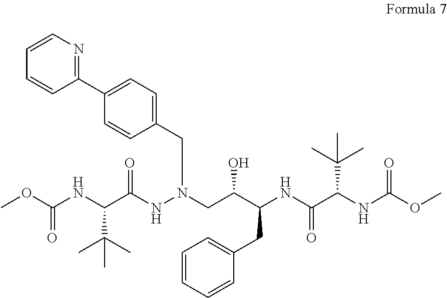

or a pharmaceutically acceptable salt, solvate, hydrate, cocrystal, or prodrug thereof. The preparation of this compound is described in U.S. Pat. Nos. 5,849,911 and 6,087,383, the disclosures of which are incorporated by reference herein. In an embodiment, the antiviral active pharmaceutical ingredient is selected from the compounds described in U.S. Pat. Nos. 5,849,911 and 6,087,383, the disclosures of which are incorporated by reference herein.

In an embodiment, the antiviral active pharmaceutical ingredient is (1R,2S,5S)—N-(4-amino-1-cyclobutyl-3,4-dioxobutan-2-yl)-3-[(2S)-2-(tert-butylcarbamoylamino)-3,3-dimethylbutanoyl]-6,6-dimethyl-3-azabicyclo[3.1.0]hexane-2-carboxamide. In a preferred embodiment, the antiviral active pharmaceutical ingredient is boceprevir. In a preferred embodiment, the antiviral active pharmaceutical ingredient has the chemical structure shown in Formula VIII:

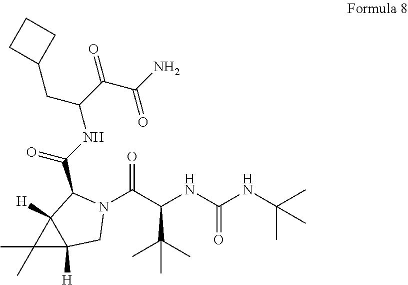 [SMILES: CC(C)(C)NC(=O)N[C@H](C(=O)N1C[C@H]2[C@@H]([C@H]1C(=O)NC(CC1CCC1)C(=O)C(N)=O)C2(C)C)C(C)(C)C]

or a pharmaceutically acceptable salt, solvate, hydrate, cocrystal, or prodrug thereof. The preparation of this compound is described in U.S. Pat. Nos. 7,772,178, 8,119,602 and RE43,298, the disclosures of which are incorporated by reference herein. In an embodiment, the antiviral active pharmaceutical ingredient is selected from the compounds described in U.S. Pat. Nos. 7,772,178, 8,119,602 and RE43,298, the disclosures of which are incorporated by reference herein.

In an embodiment, the antiviral active pharmaceutical ingredient is [(2S)-1-(4-amino-2-oxopyrimidin-1-yl)-3-hydroxypropan-2-yl]oxymethylphosphonic acid. In a preferred embodiment, the antiviral active pharmaceutical ingredient is cidofovir. In a preferred embodiment, the antiviral active pharmaceutical ingredient has the chemical structure shown in Formula 9:

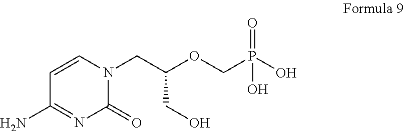 [SMILES: Nc1ccn(C[C@@H](CO)OCP(=O)(O)O)c(=O)n1]

or a pharmaceutically acceptable salt, solvate, hydrate, cocrystal, or prodrug thereof. The preparation of this compound is described in U.S. Pat. No. 5,142,051, the disclosure of which is incorporated by reference herein. In an embodiment, the antiviral active pharmaceutical ingredient is selected from the compounds described in U.S. Pat. No. 5,142,051, the disclosure of which is incorporated by reference herein.

In an embodiment, the antiviral active pharmaceutical ingredient is 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl]amino]-1,6-diphenylhexan-2-yl]carbamate. In a preferred embodiment, the antiviral active pharmaceutical ingredient is cobicistat. In a preferred embodiment, the antiviral active pharmaceutical ingredient has the chemical structure shown in Formula 10:

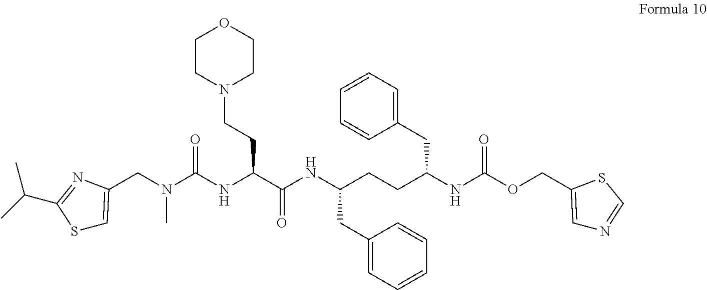 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

or a pharmaceutically acceptable salt, solvate, hydrate, cocrystal, or prodrug thereof. The preparation of this compound is described in U.S. Pat. No. 7,939,553, the disclosure of which is incorporated by reference herein. In an embodiment, the antiviral active pharmaceutical ingredient is selected from the compounds described in U.S. Pat. No. 7,939,553, the disclosure of which is incorporated by reference herein.

In an embodiment, the antiviral active pharmaceutical ingredient is (4R,12aS)—N-(2,4-difluorobenzyl)-7-hydroxy-4-methyl-6,8-dioxo-3,4,6,8,12,12a-hexahydro-2H-pyrido[1′,2′:4,5]pyrazino[2,1-b][1,3]oxazine-9-carboxamide. In a preferred embodiment, the antiviral active pharmaceutical ingredient is dolutegravir. In a preferred embodiment, the antiviral active pharmaceutical ingredient has the chemical structure shown in Formula XI:

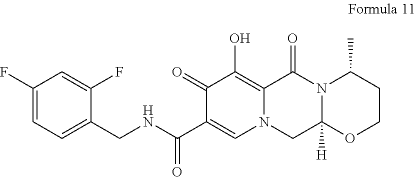 [SMILES: C[C@@H]1CCO[C@H]2Cn3cc(C(=O)NCc4ccc(F)cc4F)c(=O)c(O)c3C(=O)N12]

or a pharmaceutically acceptable salt, solvate, hydrate, cocrystal, or prodrug thereof. The preparation of this compound is described in U.S. Pat. No. 8,129,385, the disclosure of which is incorporated by reference herein. In an embodiment, the antiviral active pharmaceutical ingredient is selected from the compounds described in U.S. Pat. No. 8,129,385, the disclosure of which is incorporated by reference herein.

In an embodiment, the antiviral active pharmaceutical ingredient is [(1R,5S,6R)-2,8-dioxabicyclo[3.3.0]oct-6-yl] N-[(2S,3R)-4-[(4-aminophenyl)sulfonyl-(2-methylpropyl)amino]-3-hydroxy-1-phenyl-butan-2-yl]carbamate. In a preferred embodiment, the antiviral active pharmaceutical ingredient is darunavir. In a preferred embodiment, the antiviral active pharmaceutical ingredient has the chemical structure shown in Formula 12:

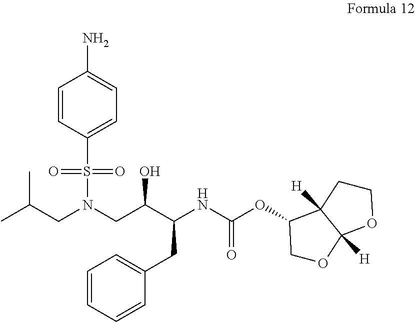 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

or a pharmaceutically acceptable salt, solvate, hydrate, cocrystal, or prodrug thereof. The preparation of this compound is described in U.S. Pat. Nos. 6,248,775; 6,335,460; 7,700,645; 5,843,946; 6,037,157; 6,703,403; 7,470,506; 8,518,987; 8,597,876; RE42,889; RE43,596; and RE43,802, the disclosures of which are incorporated by reference herein. In an embodiment, the antiviral active pharmaceutical ingredient is selected from the compounds described in U.S. Pat. Nos. 6,248,775; 6,335,460; 7,700,645; 5,843,946; 6,037,157; 6,703,403; 7,470,506; 8,518,987; 8,597,876; RE42,889; RE43,596; and RE43,802, the disclosures of which are incorporated by reference herein.

In an embodiment, the antiviral active pharmaceutical ingredient is N-[2-[4-[3-(propan-2-ylamino)pyridin-2-yl]piperazine-1-carbonyl]-1H-indol-5-yl]methanesulfonamide. In a preferred embodiment, the antiviral active pharmaceutical ingredient is delavirdine. In a preferred embodiment, the antiviral active pharmaceutical ingredient has the chemical structure shown in Formula 13:

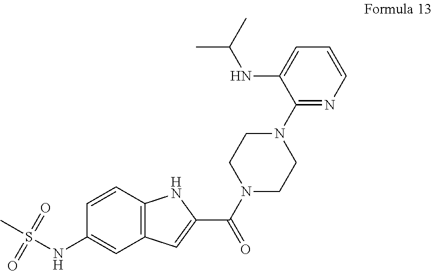 [SMILES: CC(C)Nc1cccnc1N1CCN(C(=O)c2cc3cc(NS(C)(=O)=O)ccc3[nH]2)CC1]

or a pharmaceutically acceptable salt, solvate, hydrate, cocrystal, or prodrug thereof. The preparation of this compound is described in U.S. Pat. Nos. 5,563,142 and 6,177,101, the disclosures of which are incorporated by reference herein. In an embodiment, the antiviral active pharmaceutical ingredient is selected from the compounds described in U.S. Pat. Nos. 5,563,142 and 6,177,101, the disclosures of which are incorporated by reference herein.

In an embodiment, the antiviral active pharmaceutical ingredient is 9-[(2R,5S)-5-(hydroxymethyl)oxolan-2-yl]-3H-purin-6-one. In a preferred embodiment, the antiviral active pharmaceutical ingredient is didanosine. In a preferred embodiment, the antiviral active pharmaceutical ingredient has the chemical structure shown in Formula 14:

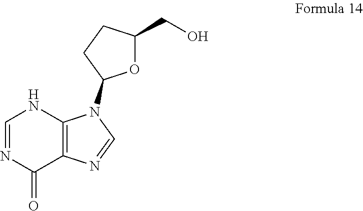 [SMILES: O=c1nc[nH]c2c1ncn2[C@H]1CC[C@@H](CO)O1]

or a pharmaceutically acceptable salt, solvate, hydrate, cocrystal, or prodrug thereof. The preparation of this compound is described in U.S. Pat. No. 5,880,106, the disclosure of which is incorporated by reference herein. In an embodiment, the antiviral active pharmaceutical ingredient is selected from the compounds described in U.S. Pat. No. 5,880,106, the disclosure of which is incorporated by reference herein.

In a preferred embodiment, the antiviral active pharmaceutical ingredient is docosan-1-ol, or a pharmaceutically acceptable salt, solvate, hydrate, cocrystal, or prodrug thereof. The preparation of this compound is described in U.S. Pat. Nos. 4,874,794 and 5,534,554, the disclosures of which are incorporated by reference herein. In an embodiment, the antiviral active pharmaceutical ingredient is selected from the compounds described in U.S. Pat. Nos. 4,874,794 and 5,534,554, the disclosures of which are incorporated by reference herein.

In a preferred embodiment, the antiviral active pharmaceutical ingredient is 5-ethyl-1-[(2R,4S,5R)-4-hydroxy-5-(hydroxymethyl)oxolan-2-yl]pyrimidine-2,4-dione. In a preferred embodiment, the antiviral active pharmaceutical ingredient is edoxudine. In a preferred embodiment, the antiviral active pharmaceutical ingredient has the chemical structure shown in Formula 15:

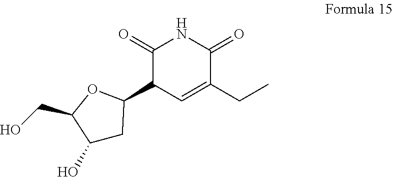 [SMILES: CCC1=CC([C@H]2C[C@H](O)[C@@H](CO)O2)C(=O)NC1=O]

or a pharmaceutically acceptable salt, solvate, hydrate, cocrystal, or prodrug thereof. The preparation of this compound is described in U.S. Pat. No. 4,267,171, the disclosure of which is incorporated by reference herein. In an embodiment, the antiviral active pharmaceutical ingredient is selected from the compounds described in U.S. Pat. No. 4,267,171, the disclosure of which is incorporated by reference herein.

In a preferred embodiment, the antiviral active pharmaceutical ingredient is (4S)-6-chloro-4-(2-cyclopropylethynyl)-4-(trifluoromethyl)-1H-3,1-benzoxazin-2-one. In a preferred embodiment, the antiviral active pharmaceutical ingredient is efavirenz. In a preferred embodiment, the antiviral active pharmaceutical ingredient has the chemical structure shown in Formula 16:

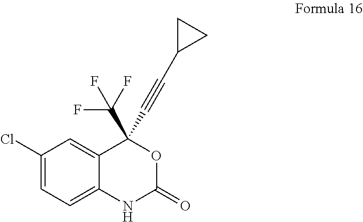 [SMILES: O=C1Nc2ccc(Cl)cc2[C@@](C#CC2CC2)(C(F)(F)F)O1]

or a pharmaceutically acceptable salt, solvate, hydrate, cocrystal, or prodrug thereof. The preparation of this compound is described in U.S. Pat. Nos. 5,811,423; 6,238,695; 5,519,021; 5,663,169; 6,555,133; 6,639,071; and 6,939,964, the disclosures of which are incorporated by reference herein. In an embodiment, the antiviral active pharmaceutical ingredient is selected from the compounds described in U.S. Pat. Nos. 5,811,423; 6,238,695; 5,519,021; 5,663,169; 6,555,133; 6,639,071; and 6,939,964, the disclosures of which are incorporated by reference herein.

In a preferred embodiment, the antiviral active pharmaceutical ingredient is 6-[(3-chloro-2-fluorophenyl)methyl]-1-[(2S)-1-hydroxy-3-methylbutan-2-yl]-7-methoxy-4-oxoquinoline-3-carboxylic acid. In a preferred embodiment, the antiviral active pharmaceutical ingredient is elvitegravir. In a preferred embodiment, the antiviral active pharmaceutical ingredient has the chemical structure shown in Formula 17:

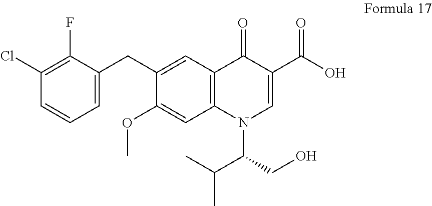 [SMILES: COc1cc2c(cc1Cc1cccc(Cl)c1F)c(=O)c(C(=O)O)cn2[C@H](CO)C(C)C]

or a pharmaceutically acceptable salt, solvate, hydrate, cocrystal, or prodrug thereof. The preparation of this compound is described in U.S. Pat. Nos. 7,635,704 and 7,176,220, the disclosures of which are incorporated by reference herein. In an embodiment, the antiviral active pharmaceutical ingredient is selected from the compounds described in U.S. Pat. Nos. 7,635,704 and 7,176,220, the disclosures of which are incorporated by reference herein.

In a preferred embodiment, the antiviral active pharmaceutical ingredient is 4-amino-5-fluoro-1-[(2R,5S)-2-(hydroxymethyl)-1,3-oxathiolan-5-yl]pyrimidin-2-one. In a preferred embodiment, the antiviral active pharmaceutical ingredient is emtricitabine. In a preferred embodiment, the antiviral active pharmaceutical ingredient has the chemical structure shown in Formula 18:

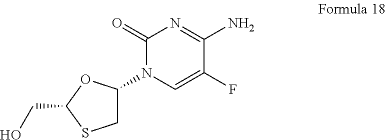 [SMILES: Nc1nc(=O)n([C@@H]2CS[C@H](CO)O2)cc1F]

or a pharmaceutically acceptable salt, solvate, hydrate, cocrystal, or prodrug thereof. The preparation of this compound is described in U.S. Pat. Nos. 6,703,396; 7,402,588; 5,814,639; 5,914,331; and 6,642,245, the disclosures of which are incorporated by reference herein. In an embodiment, the antiviral active pharmaceutical ingredient is selected from the compounds described in U.S. Pat. Nos. 6,703,396; 7,402,588; 5,814,639; 5,914,331; and 6,642,245, the disclosures of which are incorporated by reference herein.

In a preferred embodiment, the antiviral active pharmaceutical ingredient is enfuvirtide. In a preferred embodiment, the antiviral active pharmaceutical ingredient is Ac-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-NH₂, or a pharmaceutically acceptable salt, solvate, hydrate, cocrystal, or prodrug thereof. The preparation of this compound is described in U.S. Pat. Nos. 5,464,933; 6,475,491; and 6,133,418, the disclosures of which are incorporated by reference herein. In an embodiment, the antiviral active pharmaceutical ingredient is selected from the compounds described in U.S. Pat. Nos. 5,464,933; 6,475,491; and 6,133,418, the disclosures of which are incorporated by reference herein.

In a preferred embodiment, the antiviral active pharmaceutical ingredient is 2-amino-9-[(1S,3R,4S)-4-hydroxy-3-(hydroxymethyl)-2-methylidenecyclopentyl]-3H-purin-6-one. In a preferred embodiment, the antiviral active pharmaceutical ingredient is entecavir. In a preferred embodiment, the antiviral active pharmaceutical ingredient has the chemical structure shown in Formula 19:

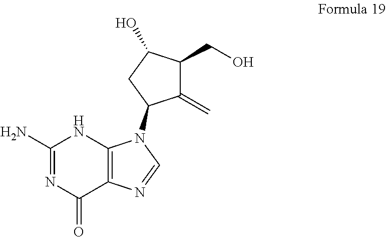 [SMILES: C=C1[C@H](CO)[C@@H](O)C[C@@H]1n1cnc2c(=O)nc(N)[nH]c21]

or a pharmaceutically acceptable salt, solvate, hydrate, cocrystal, or prodrug thereof. The preparation of this compound is described in U.S. Pat. No. 5,206,244, the disclosure of which is incorporated by reference herein. In an embodiment, the antiviral active pharmaceutical ingredient is selected from the compounds described in U.S. Pat. No. 5,206,244, the disclosure of which is incorporated by reference herein.

In a preferred embodiment, the antiviral active pharmaceutical ingredient is [2-(acetyloxymethyl)-4-(2-aminopurin-9-yl)butyl] acetate. In a preferred embodiment, the antiviral active pharmaceutical ingredient is famciclovir. In a preferred embodiment, the antiviral active pharmaceutical ingredient has the chemical structure shown in Formula 20:

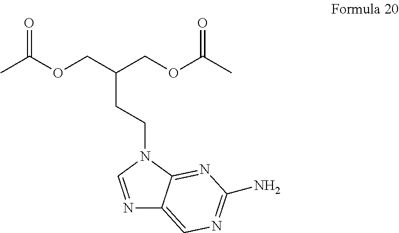 [SMILES: CC(=O)OCC(CCn1cnc2cnc(N)nc21)COC(C)=O]

or a pharmaceutically acceptable salt, solvate, hydrate, cocrystal, or prodrug thereof. The preparation of this compound is described in U.S. Pat. Nos. 5,246,937; 5,840,763; 5,866,581; 5,916,893; and 6,124,304, the disclosures of which are incorporated by reference herein. In an embodiment, the antiviral active pharmaceutical ingredient is selected from the compounds described in U.S. Pat. Nos. 5,246,937; 5,840,763; 5,866,581; 5,916,893; and 6,124,304, the disclosures of which are incorporated by reference herein.

In a preferred embodiment, the antiviral active pharmaceutical ingredient is fomivirsen. In a preferred embodiment, the antiviral active pharmaceutical ingredient is 5′-GCG TTT GCT CTT CTT CTT GCG-3′, or a pharmaceutically acceptable salt, solvate, hydrate, cocrystal, or prodrug thereof. The preparation of this compound is described in U.S. Pat. Nos. 4,689,320; 5,264,423; 5,276,019; 5,442,049; and 5,595,978, the disclosures of which are incorporated by reference herein. In an embodiment, the antiviral active pharmaceutical ingredient is selected from the compounds described in U.S. Pat. Nos. 4,689,320; 5,264,423; 5,276,019; 5,442,049; and 5,595,978, the disclosures of which are incorporated by reference herein.

In a preferred embodiment, the antiviral active pharmaceutical ingredient is {[(2R,3S)-1-[N-(2-methylpropyl)(4-aminobenzene)sulfonamido]-3-({[(3S)-oxolan-3-yloxy]carbonyl}amino)-4-phenylbutan-2-yl]oxy}phosphonic acid. In a preferred embodiment, the antiviral active pharmaceutical ingredient is fosamprenavir. In a preferred embodiment, the antiviral active pharmaceutical ingredient has the chemical structure shown in Formula 21:

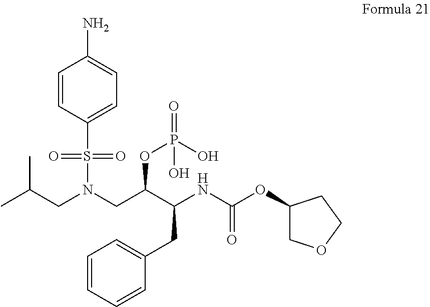 [SMILES: CC(C)CN(C[C@@H](OP(=O)(O)O)[C@H](Cc1ccccc1)NC(=O)O[C@H]1CCOC1)S(=O)(=O)c1ccc(N)cc1]

or a pharmaceutically acceptable salt, solvate, hydrate, cocrystal, or prodrug thereof. The preparation of this compound is described in U.S. Pat. Nos. 6,436,989 and 6,514,953, the disclosures of which are incorporated by reference herein. In an embodiment, the antiviral active pharmaceutical ingredient is selected from the compounds described in U.S. Pat. Nos. 6,436,989 and 6,514,953, the disclosures of which are incorporated by reference herein.

In a preferred embodiment, the antiviral active pharmaceutical ingredient is foscarnet. In a preferred embodiment, the antiviral active pharmaceutical ingredient is phosphonoformic acid, or a pharmaceutically acceptable salt, solvate, hydrate, cocrystal, or prodrug thereof. The preparation of this compound is described in U.S. Pat. No. 5,072,032, the disclosure of which is incorporated by reference herein. In an embodiment, the antiviral active pharmaceutical ingredient is selected from the compounds described in U.S. Pat. No. 5,072,032, the disclosure of which is incorporated by reference herein.

In a preferred embodiment, the antiviral active pharmaceutical ingredient is 2-amino-9-(1,3-dihydroxypropan-2-yloxymethyl)-3H-purin-6-one. In a preferred embodiment, the antiviral active pharmaceutical ingredient is ganciclovir. In a preferred embodiment, the antiviral active pharmaceutical ingredient has the chemical structure shown in Formula 22:

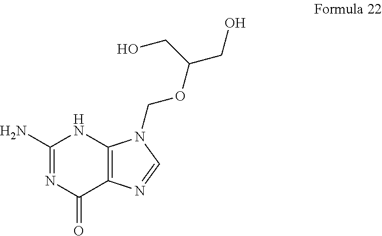 [SMILES: Nc1nc(=O)c2ncn(COC(CO)CO)c2[nH]1]

or a pharmaceutically acceptable salt, solvate, hydrate, cocrystal, or prodrug thereof. The preparation of this compound is described in U.S. Pat. No. 5,378,475, the disclosure of which is incorporated by reference herein. In an embodiment, the antiviral active pharmaceutical ingredient is selected from the compounds described in U.S. Pat. No. 5,378,475, the disclosure of which is incorporated by reference herein.

In a preferred embodiment, the antiviral active pharmaceutical ingredient is 4-amino-1-[(2R,4S,5R)-4-hydroxy-5-(hydroxymethyl)oxolan-2-yl]-5-iodopyrimidin-2-one. In a preferred embodiment, the antiviral active pharmaceutical ingredient is ibacitabine. In a preferred embodiment, the antiviral active pharmaceutical ingredient has the chemical structure shown in Formula 23:

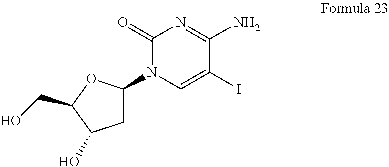 [SMILES: Nc1nc(=O)n([C@H]2C[C@H](O)[C@@H](CO)O2)cc1I]

or a pharmaceutically acceptable salt, solvate, hydrate, cocrystal, or prodrug thereof. The preparation of this compound is described in U.S. Pat. No. 4,845,081, the disclosure of which is incorporated by reference herein. In an embodiment, the antiviral active pharmaceutical ingredient is selected from the compounds described in U.S. Pat. No. 4,845,081, the disclosure of which is incorporated by reference herein.

In a preferred embodiment, the antiviral active pharmaceutical ingredient is 1-[(2R,4S,5R)-4-hydroxy-5-(hydroxymethyl)oxolan-2-yl]-5-iodopyrimidine-2,4-dione. In a preferred embodiment, the antiviral active pharmaceutical ingredient is idoxuridine. In a preferred embodiment, the antiviral active pharmaceutical ingredient has the chemical structure shown in Formula 24:

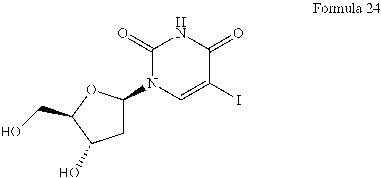 [SMILES: O=c1[nH]c(=O)n([C@H]2C[C@H](O)[C@@H](CO)O2)cc1I]

or a pharmaceutically acceptable salt, solvate, hydrate, cocrystal, or prodrug thereof. The preparation of this compound is described in U.S. Pat. No. 5,468,853, the disclosure of which is incorporated by reference herein. In an embodiment, the antiviral active pharmaceutical ingredient is selected from the compounds described in U.S. Pat. No. 5,468,853, the disclosure of which is incorporated by reference herein.

In a preferred embodiment, the antiviral active pharmaceutical ingredient is 1-(2-methylpropyl)imidazo[4,5-c]quinolin-4-amine. In a preferred embodiment, the antiviral active pharmaceutical ingredient is imiquimod. In a preferred embodiment, the antiviral active pharmaceutical ingredient has the chemical structure shown in Formula 25:

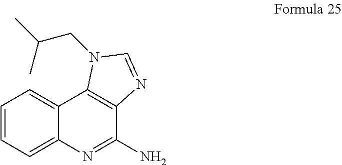 [SMILES: CC(C)Cn1cnc2c(N)nc3ccccc3c21]

or a pharmaceutically acceptable salt, solvate, hydrate, cocrystal, or prodrug thereof. The preparation of this compound is described in U.S. Pat. Nos. 4,689,338; 7,696,159; 8,236,816; 8,222,270; 8,299,109; and 8,598,196, the disclosures of which are incorporated by reference herein. In an embodiment, the antiviral active pharmaceutical ingredient is selected from the compounds described in U.S. Pat. Nos. 4,689,338; 7,696,159; 8,236,816; 8,222,270; 8,299,109; and 8,598,196, the disclosures of which are incorporated by reference herein.

In a preferred embodiment, the antiviral active pharmaceutical ingredient is (2S)-1-[(2S,4R)-4-benzyl-2-hydroxy-5-[[(1S,2R)-2-hydroxy-2,3-dihydro-1H-inden-1-yl]amino]-5-oxopentyl]-N-tert-butyl-4-(pyridin-3-ylmethyl)piperazine-2-carboxamide. In a preferred embodiment, the antiviral active pharmaceutical ingredient is indinavir. In a preferred embodiment, the antiviral active pharmaceutical ingredient has the chemical structure shown in Formula 26:

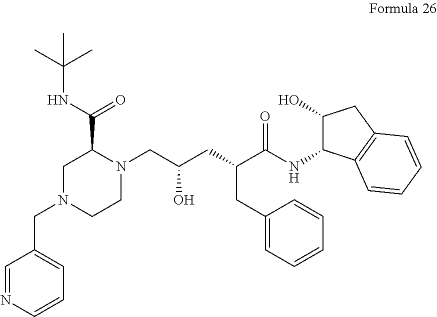 [SMILES: CC(C)(C)NC(=O)[C@@H]1CN(Cc2cccnc2)CCN1C[C@@H](O)C[C@@H](Cc1ccccc1)C(=O)N[C@H]1c2ccccc2C[C@H]1O]

or a pharmaceutically acceptable salt, solvate, hydrate, cocrystal, or prodrug thereof. The preparation of this compound is described in U.S. Pat. Nos. 5,413,999; 6,689,761; and 6,645,961, the disclosures of which are incorporated by reference herein. In an embodiment, the antiviral active pharmaceutical ingredient is selected from the compounds described in U.S. Pat. Nos. 5,413,999; 6,689,761; and 6,645,961, the disclosures of which are incorporated by reference herein.

In a preferred embodiment, the antiviral active pharmaceutical ingredient is 4-amino-1-[(2R,5S)-2-(hydroxymethyl)-1,3-oxathiolan-5-yl]pyrimidin-2-one. In a preferred embodiment, the antiviral active pharmaceutical ingredient is lamivudine. In a preferred embodiment, the antiviral active pharmaceutical ingredient has the chemical structure shown in Formula 27:

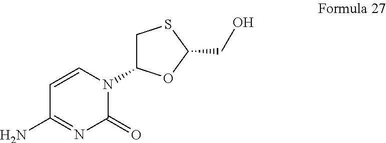 [SMILES: Nc1ccn([C@@H]2CS[C@H](CO)O2)c(=O)n1]

or a pharmaceutically acceptable salt, solvate, hydrate, cocrystal, or prodrug thereof. The preparation of this compound is described in U.S. Pat. Nos. 6,004,968; 7,119,202; 5,905,082; and RE39155, the disclosures of which are incorporated by reference herein. In an embodiment, the antiviral active pharmaceutical ingredient is selected from the compounds described in U.S. Pat. Nos. 6,004,968; 7,119,202; 5,905,082; and RE39155, the disclosures of which are incorporated by reference herein.

In a preferred embodiment, the antiviral active pharmaceutical ingredient is (2S)—N-[(2S,4S,5S)-5-[[2-(2,6-dimethylphenoxy)acetyl]amino]-4-hydroxy-1,6-diphenylhexan-2-yl]-3-methyl-2-(2-oxo-1,3-diazinan-1-yl)butanamide. In a preferred embodiment, the antiviral active pharmaceutical ingredient is lopinavir. In a preferred embodiment, the antiviral active pharmaceutical ingredient has the chemical structure shown in Formula 28:

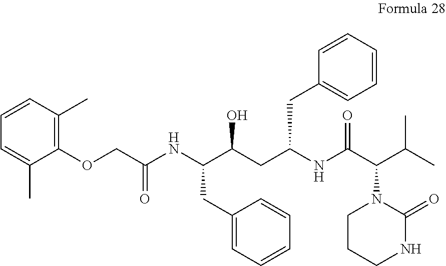 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O]

or a pharmaceutically acceptable salt, solvate, hydrate, cocrystal, or prodrug thereof. The preparation of this compound is described in U.S. Pat. No. 5,914,332, the disclosure of which is incorporated by reference herein. In an embodiment, the antiviral active pharmaceutical ingredient is selected from the compounds described in U.S. Pat. No. 5,914,332, the disclosure of which is incorporated by reference herein.

In a preferred embodiment, the antiviral active pharmaceutical ingredient is 2-(2-acetyl-5-methylanilino)-2-(2,6-dichlorophenyl)acetamide. In a preferred embodiment, the antiviral active pharmaceutical ingredient is loviride. In a preferred embodiment, the antiviral active pharmaceutical ingredient has the chemical structure shown in Formula 29:

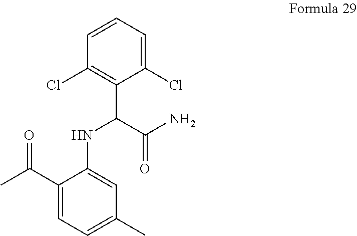 [SMILES: CC(=O)c1ccc(C)cc1NC(C(N)=O)c1c(Cl)cccc1Cl]

or a pharmaceutically acceptable salt, solvate, hydrate, cocrystal, or prodrug thereof. The preparation of this compound is described in U.S. Pat. No. 5,556,886, the disclosure of which is incorporated by reference herein. In an embodiment, the antiviral active pharmaceutical ingredient is selected from the compounds described in U.S. Pat. No. 5,556,886, the disclosure of which is incorporated by reference herein.

In a preferred embodiment, the antiviral active pharmaceutical ingredient is 4,4-difluoro-N-[(1S)-3-[(1S,5R)-3-(3-methyl-5-propan-2-yl-1,2,4-triazol-4-yl)-8-azabicyclo[3.2.1]octan-8-yl]-1-phenylpropyl]cyclohexane-1-carboxamide. In a preferred embodiment, the antiviral active pharmaceutical ingredient is maraviroc. In a preferred embodiment, the antiviral active pharmaceutical ingredient has the chemical structure shown in Formula 30:

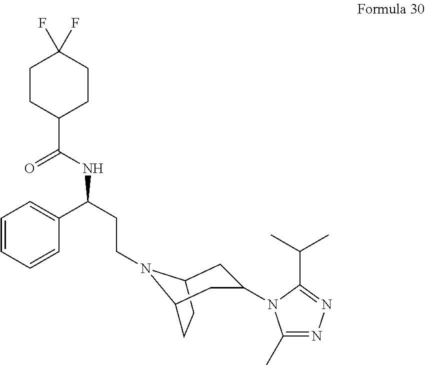 [SMILES: Cc1nnc(C(C)C)n1C1CC2CCC(C1)N2CC[C@H](NC(=O)C1CCC(F)(F)CC1)c1ccccc1]

or a pharmaceutically acceptable salt, solvate, hydrate, cocrystal, or prodrug thereof. The preparation of this compound is described in U.S. Pat. Nos. 6,586,430; 7,368,460; 6,667,314; and 7,576,097, the disclosures of which are incorporated by reference herein. In an embodiment, the antiviral active pharmaceutical ingredient is selected from the compounds described in U.S. Pat. Nos. 6,586,430; 7,368,460; 6,667,314; and 7,576,097, the disclosures of which are incorporated by reference herein.

In a preferred embodiment, the antiviral active pharmaceutical ingredient is N-(diaminomethylidene)morpholine-4-carboximidamide. In a preferred embodiment, the antiviral active pharmaceutical ingredient is moroxydine. In a preferred embodiment, the antiviral active pharmaceutical ingredient has the chemical structure shown in Formula 31:

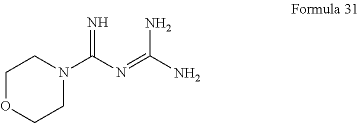 [SMILES: N=C(N=C(N)N)N1CCOCC1]

or a pharmaceutically acceptable salt, solvate, hydrate, cocrystal, or prodrug thereof. The preparation of this compound is described in U.S. Pat. Nos. 4,656,291; 4,372,954; and 4,089,957, the disclosures of which are incorporated by reference herein. In an embodiment, the antiviral active pharmaceutical ingredient is selected from the compounds described in U.S. Pat. Nos. 4,656,291; 4,372,954; and 4,089,957, the disclosures of which are incorporated by reference herein.

In a preferred embodiment, the antiviral active pharmaceutical ingredient is [(Z)-(1-methyl-2-oxoindol-3-ylidene)amino]thiourea. In a preferred embodiment, the antiviral active pharmaceutical ingredient is methisazone. In a preferred embodiment, the antiviral active pharmaceutical ingredient has the chemical structure shown in Formula 32:

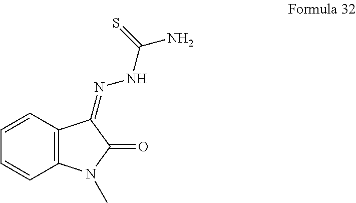 [SMILES: CN1C(=O)/C(=N\NC(N)=S)c2ccccc21]

or a pharmaceutically acceptable salt, solvate, hydrate, cocrystal, or prodrug thereof.

In a preferred embodiment, the antiviral active pharmaceutical ingredient is (3S,4aS,8aS)—N-tert-butyl-2-[(2R,3R)-2-hydroxy-3-[(3-hydroxy-2-methylbenzoyl)amino]-4-phenylsulfanylbutyl]-3,4,4a,5,6,7,8,8a-octahydro-1H-isoquinoline-3-carboxamide. In a preferred embodiment, the antiviral active pharmaceutical ingredient is nelfinavir. In a preferred embodiment, the antiviral active pharmaceutical ingredient has the chemical structure shown in Formula 33:

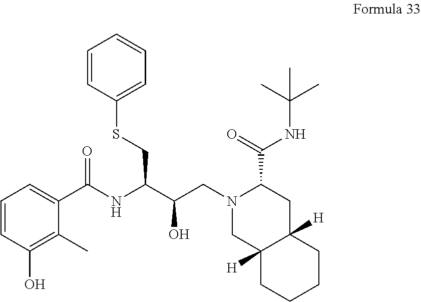 [SMILES: Cc1c(O)cccc1C(=O)N[C@@H](CSc1ccccc1)[C@H](O)CN1C[C@H]2CCCC[C@H]2C[C@H]1C(=O)NC(C)(C)C]

or a pharmaceutically acceptable salt, solvate, hydrate, cocrystal, or prodrug thereof. The preparation of this compound is described in U.S. Pat. Nos. 5,484,926; 5,952,343; and 6,162,812, the disclosures of which are incorporated by reference herein. In an embodiment, the antiviral active pharmaceutical ingredient is selected from the compounds described in U.S. Pat. Nos. 5,484,926; 5,952,343; and 6,162,812, the disclosures of which are incorporated by reference herein.

In a preferred embodiment, the antiviral active pharmaceutical ingredient is 11-cyclopropyl-4-methyl-5,11-dihydro-6H-dipyrido[3,2-b:2′,3′-e][1,4]diazepin-6-one. In a preferred embodiment, the antiviral active pharmaceutical ingredient is nevirapine. In a preferred embodiment, the antiviral active pharmaceutical ingredient has the chemical structure shown in Formula 34:

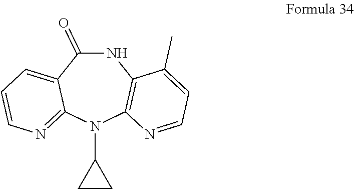 [SMILES: Cc1ccnc2c1NC(=O)c1cccnc1N2C1CC1]

or a pharmaceutically acceptable salt, solvate, hydrate, cocrystal, or prodrug thereof. The preparation of this compound is described in U.S. Pat. Nos. 5,366,972 and 8,460,704, the disclosures of which are incorporated by reference herein. In an embodiment, the antiviral active pharmaceutical ingredient is selected from the compounds described in U.S. Pat. Nos. 5,366,972 and 8,460,704, the disclosures of which are incorporated by reference herein.

In a preferred embodiment, the antiviral active pharmaceutical ingredient is ethyl (3R,4R,5S)-4-acetamido-5-amino-3-pentan-3-yloxycyclohexene-1-carboxylate. In a preferred embodiment, the antiviral active pharmaceutical ingredient is oseltamivir. In a preferred embodiment, the antiviral active pharmaceutical ingredient has the chemical structure shown in Formula 35:

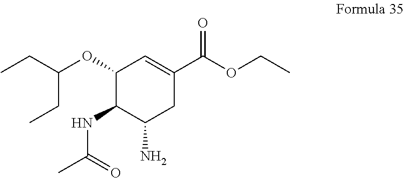 [SMILES: CCOC(=O)C1=C[C@@H](OC(CC)CC)[C@H](NC(C)=O)[C@@H](N)C1]

or a pharmaceutically acceptable salt, solvate, hydrate, cocrystal, or prodrug thereof. The preparation of this compound is described in U.S. Pat. Nos. 5,763,483; 5,866,601; and 5,952,375 the disclosures of which are incorporated by reference herein. In an embodiment, the antiviral active pharmaceutical ingredient is selected from the compounds described in U.S. Pat. Nos. 5,763,483; 5,866,601; and 5,952,375, the disclosures of which are incorporated by reference herein.

In a preferred embodiment, the antiviral active pharmaceutical ingredient is 2-amino-9-[4-hydroxy-3-(hydroxymethyl)butyl]-3H-purin-6-one. In a preferred embodiment, the antiviral active pharmaceutical ingredient is penciclovir. In a preferred embodiment, the antiviral active pharmaceutical ingredient has the chemical structure shown in Formula 36:

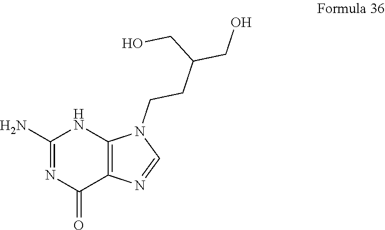 [SMILES: Nc1nc(=O)c2ncn(CCC(CO)CO)c2[nH]1]

or a pharmaceutically acceptable salt, solvate, hydrate, cocrystal, or prodrug thereof. The preparation of this compound is described in U.S. Pat. Nos. 5,075,445; 6,579,981; 5,840,763; 6,124,304; 5,916,893; 6,469,015; and 5,866,581, the disclosures of which are incorporated by reference herein. In an embodiment, the antiviral active pharmaceutical ingredient is selected from the compounds described in U.S. Pat. Nos. 5,075,445; 6,579,981; 5,840,763; 6,124,304; 5,916,893; 6,469,015; and 5,866,581, the disclosures of which are incorporated by reference herein.

In a preferred embodiment, the antiviral active pharmaceutical ingredient is (1S,2S,3 S,4R)-3-[(1 S)-1-acetamido-2-ethylbutyl]-4-(diaminomethylideneamino)-2-hydroxycyclopentane-1-carboxylic acid. In a preferred embodiment, the antiviral active pharmaceutical ingredient is peramivir. In a preferred embodiment, the antiviral active pharmaceutical ingredient has the chemical structure shown in Formula 37:

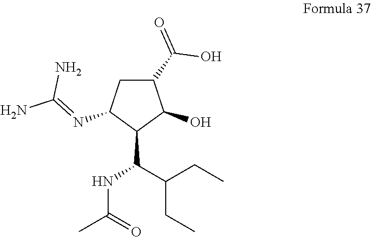

or a pharmaceutically acceptable salt, solvate, hydrate, cocrystal, or prodrug thereof. The preparation of this compound is described in U.S. Patent Application Nos. 2011/0065795 and 2013/0331604, the disclosures of which are incorporated by reference herein. In an embodiment, the antiviral active pharmaceutical ingredient is selected from the compounds described in U.S. Patent Application Nos. 2011/0065795 and 2013/0331604, the disclosures of which are incorporated by reference herein.

In a preferred embodiment, the antiviral active pharmaceutical ingredient is 3-[3,5-dimethyl-4-[3-(3-methyl-1,2-oxazol-5-yl)propoxy]phenyl]-5-(trifluoromethyl)-1,2,4-oxadiazole. In a preferred embodiment, the antiviral active pharmaceutical ingredient is pleconaril. In a preferred embodiment, the antiviral active pharmaceutical ingredient has the chemical structure shown in Formula 38:

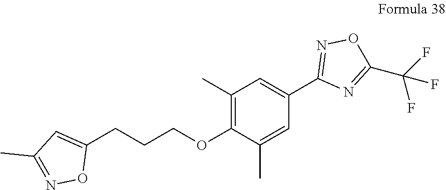 [SMILES: Cc1cc(CCCOc2c(C)cc(-c3noc(C(F)(F)F)n3)cc2C)on1]

or a pharmaceutically acceptable salt, solvate, hydrate, cocrystal, or prodrug thereof. The preparation of this compound is described in U.S. Pat. Nos. 5,464,848 and 7,060,708, the disclosures of which are incorporated by reference herein. In an embodiment, the antiviral active pharmaceutical ingredient is selected from the compounds described in U.S. Pat. Nos. 5,464,848 and 7,060,708, the disclosures of which are incorporated by reference herein.

In a preferred embodiment, the antiviral active pharmaceutical ingredient is (5R,5aR,8aR,9R)-5-hydroxy-9-(3,4,5-trimethoxyphenyl)-5a,6,8a,9-tetrahydro-5H-[2]benzofuro[5,6-f][1,3]benzodioxol-8-one. In a preferred embodiment, the antiviral active pharmaceutical ingredient is podophyllotoxin. In a preferred embodiment, the antiviral active pharmaceutical ingredient has the chemical structure shown in Formula 39:

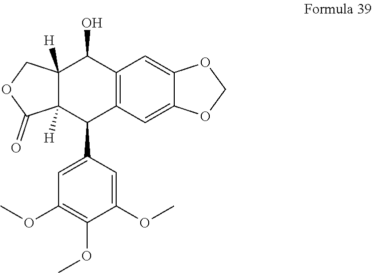 [SMILES: COc1cc([C@@H]2c3cc4c(cc3[C@H](O)[C@H]3COC(=O)[C@H]23)OCO4)cc(OC)c1OC]

or a pharmaceutically acceptable salt, solvate, hydrate, cocrystal, or prodrug thereof. The preparation of this compound is described in U.S. Pat. Nos. 5,057,616 and 5,315,016, the disclosures of which are incorporated by reference herein. In an embodiment, the antiviral active pharmaceutical ingredient is selected from the compounds described in U.S. Pat. Nos. 5,057,616 and 5,315,016, the disclosures of which are incorporated by reference herein.

In a preferred embodiment, the antiviral active pharmaceutical ingredient is N-[2-[4-[(4-fluorophenyl)methylcarbamoyl]-5-hydroxy-1-methyl-6-oxopyrimidin-2-yl]propan-2-yl]-5-methyl-1,3,4-oxadiazole-2-carboxamide. In a preferred embodiment, the antiviral active pharmaceutical ingredient is raltegravir. In a preferred embodiment, the antiviral active pharmaceutical ingredient has the chemical structure shown in Formula 40:

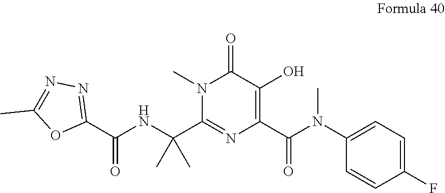 [SMILES: Cc1nnc(C(=O)NC(C)(C)c2nc(C(=O)N(C)c3ccc(F)cc3)c(O)c(=O)n2C)o1]

or a pharmaceutically acceptable salt, solvate, hydrate, cocrystal, or prodrug thereof. The preparation of this compound is described in U.S. Pat. Nos. 7,169,780; 7,217,713; 7,754,731; and 7,435,734, the disclosures of which are incorporated by reference herein. In an embodiment, the antiviral active pharmaceutical ingredient is selected from the compounds described in U.S. Pat. Nos. 7,169,780; 7,217,713; 7,754,731; and 7,435,734, the disclosures of which are incorporated by reference herein.

In a preferred embodiment, the antiviral active pharmaceutical ingredient is 1-[(2R,3R,4S,5R)-3,4-dihydroxy-5-(hydroxymethyl)oxolan-2-yl]-1,2,4-triazole-3-carboxamide. In a preferred embodiment, the antiviral active pharmaceutical ingredient is ribavirin. In a preferred embodiment, the antiviral active pharmaceutical ingredient has the chemical structure shown in Formula 41:

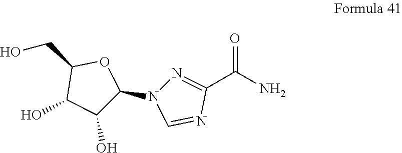 [SMILES: NC(=O)c1ncn([C@@H]2O[C@H](CO)[C@@H](O)[C@H]2O)n1]

or a pharmaceutically acceptable salt, solvate, hydrate, cocrystal, or prodrug thereof. The preparation of this compound is described in U.S. Pat. Nos. 6,177,074; 6,790,837; 6,150,337; 6,172,046; 6,461,605; 6,472,373; and 6,524,570, the disclosures of which are incorporated by reference herein. In an embodiment, the antiviral active pharmaceutical ingredient is selected from the compounds described in U.S. Pat. Nos. 6,177,074; 6,790,837; 6,150,337; 6,172,046; 6,461,605; 6,472,373; and 6,524,570, the disclosures of which are incorporated by reference herein.

In a preferred embodiment, the antiviral active pharmaceutical ingredient is 4-[[4-[4-[(E)-2-cyanoethenyl]-2,6-dimethylanilino]pyrimidin-2-yl]amino]benzonitrile. In a preferred embodiment, the antiviral active pharmaceutical ingredient is rilpivirine. In a preferred embodiment, the antiviral active pharmaceutical ingredient has the chemical structure shown in Formula 42:

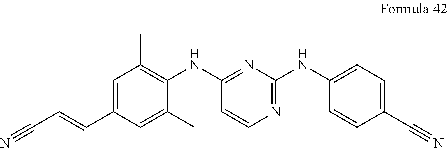 [SMILES: Cc1cc(/C=C/C#N)cc(C)c1Nc1ccnc(Nc2ccc(C#N)cc2)n1]

or a pharmaceutically acceptable salt, solvate, hydrate, cocrystal, or prodrug thereof. The preparation of this compound is described in U.S. Pat. Nos. 6,838,464, 7,067,522, 7,125,879, 7,638,522, 8,080,551, and 8,101,629, the disclosures of which are incorporated by reference herein. In an embodiment, the antiviral active pharmaceutical ingredient is selected from the compounds described in U.S. Pat. Nos. 6,838,464, 7,067,522, 7,125,879, 7,638,522, 8,080,551, and 8,101,629, the disclosures of which are incorporated by reference herein.

In a preferred embodiment, the antiviral active pharmaceutical ingredient is 1-(1-adamantyl)ethanamine. In a preferred embodiment, the antiviral active pharmaceutical ingredient is rimantadine. In a preferred embodiment, the antiviral active pharmaceutical ingredient has the chemical structure shown in Formula 43:

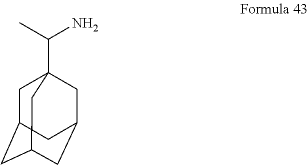

or a pharmaceutically acceptable salt, solvate, hydrate, cocrystal, or prodrug thereof. The preparation of this compound is described in U.S. Pat. Nos. 4,027,035; 4,551,552; and 3,352,912, the disclosures of which are incorporated by reference herein. In an embodiment, the antiviral active pharmaceutical ingredient is selected from the compounds described in U.S. Pat. Nos. 4,027,035; 4,551,552; and 3,352,912, the disclosures of which are incorporated by reference herein.

In a preferred embodiment, the antiviral active pharmaceutical ingredient is 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate. In a preferred embodiment, the antiviral active pharmaceutical ingredient is ritonavir. In a preferred embodiment, the antiviral active pharmaceutical ingredient has the chemical structure shown in Formula 44:

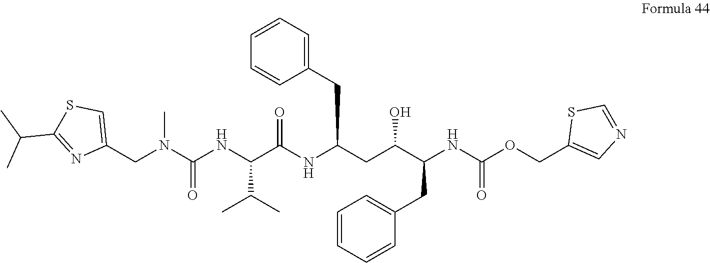 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

or a pharmaceutically acceptable salt, solvate, hydrate, cocrystal, or prodrug thereof. The preparation of this compound is described in U.S. Pat. Nos. 5,541,206; 7,364,752; 5,484,801; 5,635,523; 5,648,497; 5,674,882; 5,948,436; 6,037,157; 6,232,333; 6,703,403; 7,141,593; 7,148,359; 7,432,294; 8,268,349; 8,399,015; 8,470,347; and 8,691,878, the disclosures of which are incorporated by reference herein. In an embodiment, the antiviral active pharmaceutical ingredient is selected from the compounds described in U.S. Pat. Nos. 5,541,206; 7,364,752; 5,484,801; 5,635,523; 5,648,497; 5,674,882; 5,948,436; 6,037,157; 6,232,333; 6,703,403; 7,141,593; 7,148,359; 7,432,294; 8,268,349; 8,399,015; 8,470,347; and 8,691,878, the disclosures of which are incorporated by reference herein.

In a preferred embodiment, the antiviral active pharmaceutical ingredient is (2S)—N-[(2S,3R)-4-[(3S,4aS,8aS)-3-(tert-butylcarbamoyl)-3,4,4a,5,6,7,8,8a-octahydro-1H-isoquinolin-2-yl]-3-hydroxy-1-phenylbutan-2-yl]-2-(quinoline-2-carbonylamino)butanediamide. In a preferred embodiment, the antiviral active pharmaceutical ingredient is saquinavir. In a preferred embodiment, the antiviral active pharmaceutical ingredient has the chemical structure shown in Formula 45:

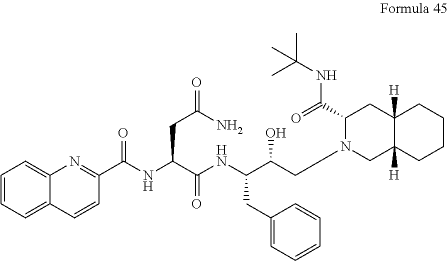 [SMILES: CC(C)(C)NC(=O)[C@@H]1C[C@@H]2CCCC[C@@H]2CN1C[C@@H](O)[C@H](Cc1ccccc1)NC(=O)[C@H](CC(N)=O)NC(=O)c1ccc2ccccc2n1]

or a pharmaceutically acceptable salt, solvate, hydrate, cocrystal, or prodrug thereof. The preparation of this compound is described in U.S. Pat. Nos. 5,196,438 and 6,352,717, the disclosures of which are incorporated by reference herein. In an embodiment, the antiviral active pharmaceutical ingredient is selected from the compounds described in U.S. Pat. Nos. 5,196,438 and 6,352,717, the disclosures of which are incorporated by reference herein.

In a preferred embodiment, the antiviral active pharmaceutical ingredient is propan-2-yl (2S)-2-[[[(2R,3R,4R,5R)-5-(2,4-dioxopyrimidin-1-yl)-4-fluoro-3-hydroxy-4-methyloxolan-2-yl]methoxy-phenoxyphosphoryl]amino]propanoate. In a preferred embodiment, the antiviral active pharmaceutical ingredient is sofosbuvir. In a preferred embodiment, the antiviral active pharmaceutical ingredient has the chemical structure shown in Formula 46:

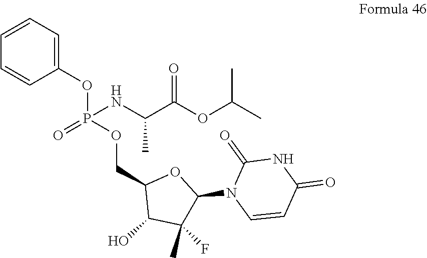 [SMILES: CC(C)OC(=O)[C@H](C)NP(=O)(OC[C@H]1O[C@@H](n2ccc(=O)[nH]c2=O)[C@](C)(F)[C@@H]1O)Oc1ccccc1]

or a pharmaceutically acceptable salt, solvate, hydrate, cocrystal, or prodrug thereof. The preparation of this compound is described in U.S. Patent Application No. 2010/0298257, the disclosure of which is incorporated by reference herein. In an embodiment, the antiviral active pharmaceutical ingredient is selected from the compounds described in U.S. Patent Application No. 2010/0298257, the disclosure of which is incorporated by reference herein.

In a preferred embodiment, the antiviral active pharmaceutical ingredient is 1-[(2R,5S)-5-(hydroxymethyl)-2,5-dihydrofuran-2-yl]-5-methylpyrimidine-2,4-dione. In a preferred embodiment, the antiviral active pharmaceutical ingredient is stavudine. In a preferred embodiment, the antiviral active pharmaceutical ingredient has the chemical structure shown in Formula 47:

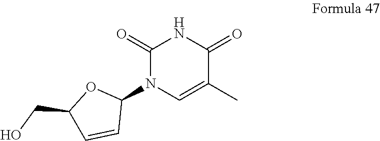 [SMILES: Cc1cn([C@H]2C=C[C@@H](CO)O2)c(=O)[nH]c1=O]

or a pharmaceutically acceptable salt, solvate, hydrate, cocrystal, or prodrug thereof. The preparation of this compound is described in U.S. Pat. Nos. 7,135,465; 8,026,356; and 5,130,421, the disclosures of which are incorporated by reference herein. In an embodiment, the antiviral active pharmaceutical ingredient is selected from the compounds described in U.S. Pat. Nos. 7,135,465; 8,026,356; and 5,130,421, the disclosures of which are incorporated by reference herein.

In a preferred embodiment, the antiviral active pharmaceutical ingredient is (3 S,3 aS,6aR)-2-[(2S)-2-[[(2S)-2-cyclohexyl-2-(pyrazine-2-carbonylamino)acetyl]amino]-3,3-dimethylbutanoyl]-N-[(3 S)-1-(cyclopropylamino)-1,2-dioxohexan-3-yl]-3,3a,4,5,6,6a-hexahydro-1H-cyclopenta[c]pyrrole-3-carboxamide. In a preferred embodiment, the antiviral active pharmaceutical ingredient is telaprevir. In a preferred embodiment, the antiviral active pharmaceutical ingredient has the chemical structure shown in Formula 48:

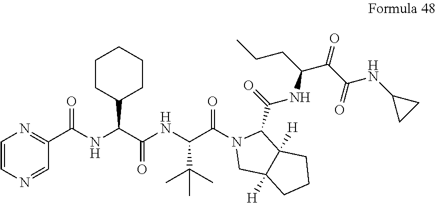 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

or a pharmaceutically acceptable salt, solvate, hydrate, cocrystal, or prodrug thereof. The preparation of this compound is described in U.S. Pat. Nos. 7,820,671; 8,529,882; and 8,431,615, the disclosures of which are incorporated by reference herein. In an embodiment, the antiviral active pharmaceutical ingredient is selected from the compounds described in U.S. Pat. Nos. 7,820,671; 8,529,882; and 8,431,615, the disclosures of which are incorporated by reference herein.

In a preferred embodiment, the antiviral active pharmaceutical ingredient is [(2R)-1-(6-aminopurin-9-yl)propan-2-yl]oxymethylphosphonic acid. In a preferred embodiment, the antiviral active pharmaceutical ingredient is tenofovir. In a preferred embodiment, the antiviral active pharmaceutical ingredient has the chemical structure shown in Formula 49:

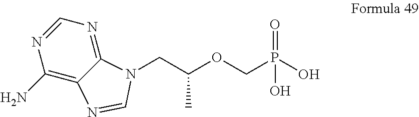 [SMILES: C[C@H](Cn1cnc2c(N)ncnc21)OCP(=O)(O)O]

or a pharmaceutically acceptable salt, solvate, hydrate, cocrystal, or prodrug thereof. The preparation of this compound is described in U.S. Pat. Nos. 5,922,695 and 8,049,009, the disclosures of which are incorporated by reference herein. In an embodiment, the antiviral active pharmaceutical ingredient is selected from the compounds described in U.S. Pat. Nos. 5,922,695 and 8,049,009, the disclosures of which are incorporated by reference herein.

In a preferred embodiment, the antiviral active pharmaceutical ingredient is [[(2R)-1-(6-aminopurin-9-yl)propan-2-yl]oxymethyl-(propan-2-yloxycarbonyloxymethoxy)phosphoryl] oxymethyl propan-2-yl carbonate. In a preferred embodiment, the antiviral active pharmaceutical ingredient is tenofovir disoproxil. In a preferred embodiment, the antiviral active pharmaceutical ingredient has the chemical structure shown in Formula 50:

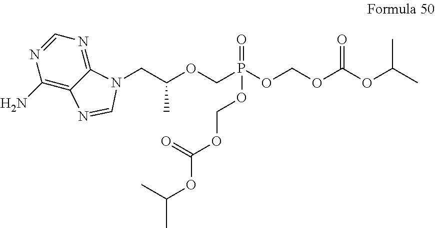 [SMILES: CC(C)OC(=O)OCOP(=O)(CO[C@H](C)Cn1cnc2c(N)ncnc21)OCOC(=O)OC(C)C]

or a pharmaceutically acceptable salt, solvate, hydrate, cocrystal, or prodrug thereof. In a preferred embodiment, the antiviral active pharmaceutical ingredient is tenofovir disoproxil difumarate. The preparation of this compound is described in U.S. Patent Application Nos. 2011/0009368 and 2011/0112292 and U.S. Pat. Nos. 5,935,946; 5,922,695; 5,977,089; 6,043,230; and 6,069,249, the disclosures of which are incorporated by reference herein. In an embodiment, the antiviral active pharmaceutical ingredient is selected from the compounds described in U.S. Patent Application Nos. 2011/0009368 and 2011/0112292 and U.S. Pat. Nos. 5,935,946; 5,922,695; 5,977,089; 6,043,230; and 6,069,249, the disclosures of which are incorporated by reference herein.

In a preferred embodiment, the antiviral active pharmaceutical ingredient is N-[3-[(1R)-1-[(2R)-4-hydroxy-6-oxo-2-(2-phenylethyl)-2-propyl-3H-pyran-5-yl]propyl]phenyl]-5-(trifluoromethyl)pyridine-2-sulfonamide. In a preferred embodiment, the antiviral active pharmaceutical ingredient is tipranavir. In a preferred embodiment, the antiviral active pharmaceutical ingredient has the chemical structure shown in Formula 51:

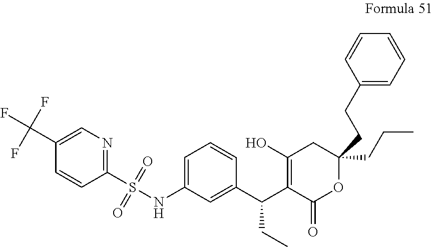 [SMILES: CCC[C@@]1(CCc2ccccc2)CC(O)=C([C@H](CC)c2cccc(NS(=O)(=O)c3ccc(C(F)(F)F)cn3)c2)C(=O)O1]

or a pharmaceutically acceptable salt, solvate, hydrate, cocrystal, or prodrug thereof. The preparation of this compound is described in U.S. Pat. Nos. 6,147,095; 6,169,181; 7,002,017; 5,852,195; and 6,231,887, the disclosures of which are incorporated by reference herein. In an embodiment, the antiviral active pharmaceutical ingredient is selected from the compounds described in U.S. Pat. Nos. 6,147,095; 6,169,181; 7,002,017; 5,852,195; and 6,231,887, the disclosures of which are incorporated by reference herein.

In a preferred embodiment, the antiviral active pharmaceutical ingredient is 1-[(2R,4S,5R)-4-hydroxy-5-(hydroxymethyl)oxolan-2-yl]-5-(trifluoromethyl)pyrimidine-2,4-dione. In a preferred embodiment, the antiviral active pharmaceutical ingredient is trifluridine. In a preferred embodiment, the antiviral active pharmaceutical ingredient has the chemical structure shown in Formula 52:

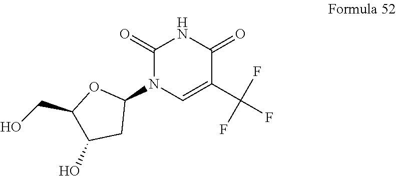 [SMILES: O=c1[nH]c(=O)n([C@H]2C[C@H](O)[C@@H](CO)O2)cc1C(F)(F)F]

or a pharmaceutically acceptable salt, solvate, hydrate, cocrystal, or prodrug thereof. The preparation of this compound is described in U.S. Pat. Nos. 3,201,387 and 3,531,464, the disclosures of which are incorporated by reference herein. In an embodiment, the antiviral active pharmaceutical ingredient is selected from the compounds described in U.S. Pat. Nos. 3,201,387 and 3,531,464, the disclosures of which are incorporated by reference herein.

In a preferred embodiment, the antiviral active pharmaceutical ingredient is N-(1-adamantyl)-2-[2-(dimethylamino)ethoxy]acetamide. In a preferred embodiment, the antiviral active pharmaceutical ingredient is tromantadine. In a preferred embodiment, the antiviral active pharmaceutical ingredient has the chemical structure shown in Formula 53:

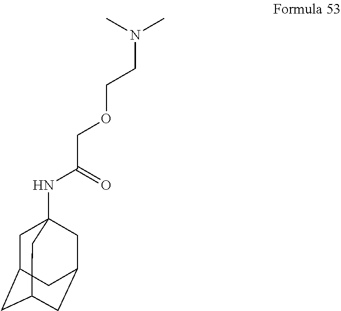 [SMILES: CN(C)CCOCC(=O)NC12CC3CC(CC(C3)C1)C2]

or a pharmaceutically acceptable salt, solvate, hydrate, cocrystal, or prodrug thereof. The preparation of this compound is described in U.S. Patent Application No. 2013/0072553, the disclosure of which is incorporated by reference herein. In an embodiment, the antiviral active pharmaceutical ingredient is selected from the compounds described in U.S. Patent Application No. 2013/0072553, the disclosure of which is incorporated by reference herein.

In a preferred embodiment, the antiviral active pharmaceutical ingredient is 2-[(2-amino-6-oxo-3H-purin-9-yl)methoxy]ethyl (2S)-2-amino-3-methylbutanoate. In a preferred embodiment, the antiviral active pharmaceutical ingredient is valacyclovir. In a preferred embodiment, the antiviral active pharmaceutical ingredient has the chemical structure shown in Formula 54:

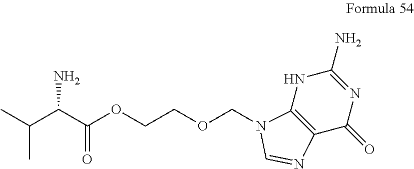 [SMILES: CC(C)[C@H](N)C(=O)OCCOCn1cnc2c(=O)nc(N)[nH]c21]

or a pharmaceutically acceptable salt, solvate, hydrate, cocrystal, or prodrug thereof. The preparation of this compound is described in U.S. Pat. Nos. 4,957,924; 5,879,706; 6,849,737; and 6,107,302, the disclosures of which are incorporated by reference herein. In an embodiment, the antiviral active pharmaceutical ingredient is selected from the compounds described in U.S. Pat. Nos. 4,957,924; 5,879,706; 6,849,737; and 6,107,302, the disclosures of which are incorporated by reference herein.

In a preferred embodiment, the antiviral active pharmaceutical ingredient is [2-[(2-amino-6-oxo-3H-purin-9-yl)methoxy]-3-hydroxypropyl] (2S)-2-amino-3-methylbutanoate. In a preferred embodiment, the antiviral active pharmaceutical ingredient is valganciclovir. In a preferred embodiment, the antiviral active pharmaceutical ingredient has the chemical structure shown in Formula 55:

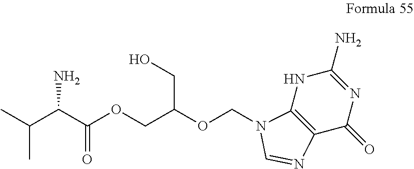 [SMILES: CC(C)[C@H](N)C(=O)OCC(CO)OCn1cnc2c(=O)nc(N)[nH]c21]

or a pharmaceutically acceptable salt, solvate, hydrate, cocrystal, or prodrug thereof. The preparation of this compound is described in U.S. Pat. No. 6,083,953 and U.S. Patent Application No. 2007/0292499, the disclosures of which are incorporated by reference herein. In an embodiment, the antiviral active pharmaceutical ingredient is selected from the compounds described in U.S. Pat. No. 6,083,953 and U.S. Patent Application No. 2007/0292499, the disclosures of which are incorporated by reference herein.

In a preferred embodiment, the antiviral active pharmaceutical ingredient is (4,6-dimethylpyrimidin-5-yl)-[4-[(3S)-4-[(1R)-2-methoxy-1-[4-(trifluoromethyl)phenyl]ethyl]-3-methylpiperazin-1-yl]-4-methylpiperidin-1-yl]methanone. In a preferred embodiment, the antiviral active pharmaceutical ingredient is vicriviroc. In a preferred embodiment, the antiviral active pharmaceutical ingredient has the chemical structure shown in Formula 56:

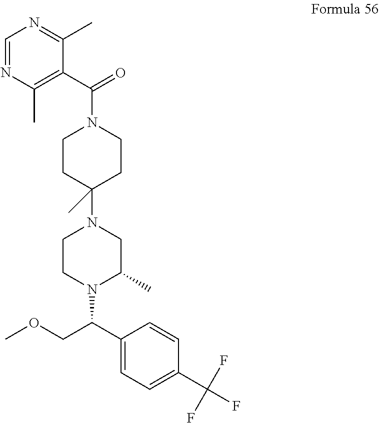 [SMILES: COC[C@@H](c1ccc(C(F)(F)F)cc1)N1CCN(C2(C)CCN(C(=O)c3c(C)ncnc3C)CC2)C[C@@H]1C]

or a pharmaceutically acceptable salt, solvate, hydrate, cocrystal, or prodrug thereof. The preparation of this compound is described in U.S. Pat. Nos. 7,534,884; 6,391,865; 6,943,251; and 6,689,765, the disclosures of which are incorporated by reference herein. In an embodiment, the antiviral active pharmaceutical ingredient is selected from the compounds described in U.S. Pat. Nos. 7,534,884; 6,391,865; 6,943,251; and 6,689,765, the disclosures of which are incorporated by reference herein.

In a preferred embodiment, the antiviral active pharmaceutical ingredient is (2R,3S,4S,5R)-2-(6-aminopurin-9-yl)-5-(hydroxymethyl)oxolane-3,4-diol. In a preferred embodiment, the antiviral active pharmaceutical ingredient is vidarabine. In a preferred embodiment, the antiviral active pharmaceutical ingredient has the chemical structure shown in Formula 57:

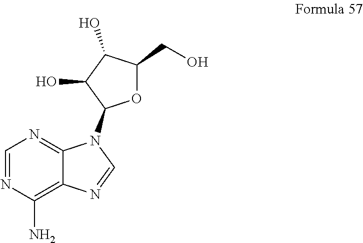 [SMILES: Nc1ncnc2c1ncn2[C@@H]1O[C@H](CO)[C@@H](O)[C@@H]1O]

or a pharmaceutically acceptable salt, solvate, hydrate, cocrystal, or prodrug thereof. The preparation of this compound is described in U.S. Pat. Nos. 3,703,507; 4,123,609; and 5,506,352, the disclosures of which are incorporated by reference herein. In an embodiment, the antiviral active pharmaceutical ingredient is selected from the compounds described in U.S. Pat. Nos. 3,703,507; 4,123,609; and 5,506,352, the disclosures of which are incorporated by reference herein.

In a preferred embodiment, the antiviral active pharmaceutical ingredient is 1-[(2R,3R,4S,5R)-3,4-dihydroxy-5-(hydroxymethyl)oxolan-2-yl]-1,2,4-triazole-3-carboximidamide. In a preferred embodiment, the antiviral active pharmaceutical ingredient is viramidine or taribavirin. In a preferred embodiment, the antiviral active pharmaceutical ingredient has the chemical structure shown in Formula 58:

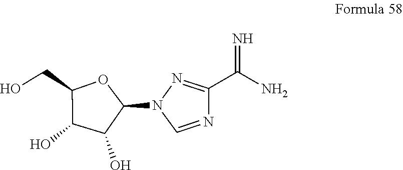 [SMILES: N=C(N)c1ncn([C@@H]2O[C@H](CO)[C@@H](O)[C@H]2O)n1]

or a pharmaceutically acceptable salt, solvate, hydrate, cocrystal, or prodrug thereof. The preparation of this compound is described in U.S. Pat. Nos. 4,925,930 and 7,638,496, the disclosures of which are incorporated by reference herein. In an embodiment, the antiviral active pharmaceutical ingredient is selected from the compounds described in U.S. Pat. Nos. 4,925,930 and 7,638,496, the disclosures of which are incorporated by reference herein.

In a preferred embodiment, the antiviral active pharmaceutical ingredient is 4-amino-1-[(2R,5S)-5-(hydroxymethyl)oxolan-2-yl]pyrimidin-2-one. In a preferred embodiment, the antiviral active pharmaceutical ingredient is zalcitabine. In a preferred embodiment, the antiviral active pharmaceutical ingredient has the chemical structure shown in Formula 59:

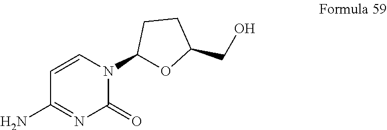 [SMILES: Nc1ccn([C@H]2CC[C@@H](CO)O2)c(=O)n1]

or a pharmaceutically acceptable salt, solvate, hydrate, cocrystal, or prodrug thereof. The preparation of this compound is described in U.S. Pat. Nos. 4,879,277 and 5,028,595, the disclosures of which are incorporated by reference herein. In an embodiment, the antiviral active pharmaceutical ingredient is selected from the compounds described in U.S. Pat. Nos. 4,879,277 and 5,028,595, the disclosures of which are incorporated by reference herein.

In a preferred embodiment, the antiviral active pharmaceutical ingredient is (2R,3R,4S)-3-acetamido-4-(diaminomethylideneamino)-2-[(1R,2R)-1,2,3-trihydroxypropyl]-3,4-dihydro-2H-pyran-6-carboxylic acid. In a preferred embodiment, the antiviral active pharmaceutical ingredient is zanamivir. In a preferred embodiment, the antiviral active pharmaceutical ingredient has the chemical structure shown in Formula 60:

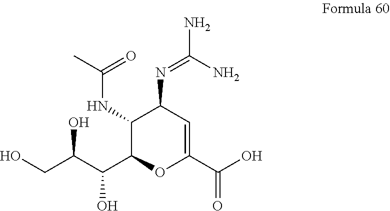 [SMILES: CC(=O)N[C@H]1[C@H]([C@H](O)[C@H](O)CO)OC(C(=O)O)=C[C@@H]1N=C(N)N]

or a pharmaceutically acceptable salt, solvate, hydrate, cocrystal, or prodrug thereof. The preparation of this compound is described in U.S. Pat. Nos. 5,360,817; 6,294,572; and 5,648,379, and U.S. Patent Application No. 2011/0257418, the disclosures of which are incorporated by reference herein. In an embodiment, the antiviral active pharmaceutical ingredient is selected from the compounds described in U.S. Pat. Nos. 5,360,817; 6,294,572; and 5,648,379, and U.S. Patent Application No. 2011/0257418, the disclosures of which are incorporated by reference herein.

In a preferred embodiment, the antiviral active pharmaceutical ingredient is 1-[(2R,4S,5S)-4-azido-5-(hydroxymethyl)oxolan-2-yl]-5-methylpyrimidine-2,4-dione. In a preferred embodiment, the antiviral active pharmaceutical ingredient is zidovudine. In a preferred embodiment, the antiviral active pharmaceutical ingredient has the chemical structure shown in Formula 61:

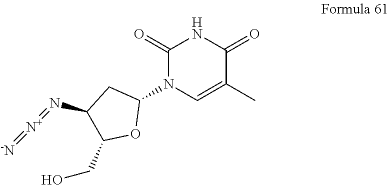

or a pharmaceutically acceptable salt, solvate, hydrate, cocrystal, or prodrug thereof. The preparation of this compound is described in U.S. Pat. No. 4,724,232, the disclosure of which is incorporated by reference herein. In an embodiment, the antiviral active pharmaceutical ingredient is selected from the compounds described in U.S. Pat. No. 4,724,232, the disclosure of which is incorporated by reference herein.

In a preferred embodiment, the antiviral active pharmaceutical ingredient is (2S,3S,4R,5S)-2-[5,6-dichloro-2-(propan-2-ylamino)benzimidazol-1-yl]-5-(hydroxymethyl)oxolane-3,4-diol. In a preferred embodiment, the antiviral active pharmaceutical ingredient is maribavir. In a preferred embodiment, the antiviral active pharmaceutical ingredient has the chemical structure shown in Formula 62:

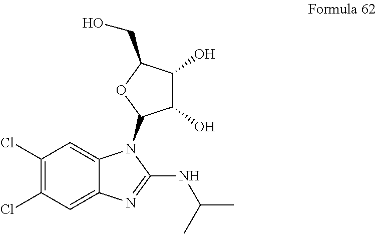

or a pharmaceutically acceptable salt, solvate, hydrate, cocrystal, or prodrug thereof. The preparation of this compound is described in U.S. Pat. Nos. 6,469,160; 6,204,249; 6,617,315; 6,482,939; 6,307,043; and 7,858,773, and U.S. Patent Application Nos. 2010/0179101 and 2011/0118203, the disclosures of which are incorporated by reference herein. In an embodiment, the antiviral active pharmaceutical ingredient is selected from the compounds described in U.S. Pat. Nos. 6,469,160; 6,204,249; 6,617,315; 6,482,939; 6,307,043; and 7,858,773, and U.S. Patent Application Nos. 2010/0179101 and 2011/0118203, the disclosures of which are incorporated by reference herein.

In a preferred embodiment, the antiviral active pharmaceutical ingredient is a combination of [(1S,4R)-4-[2-amino-6-(cyclopropylamino)purin-9-yl]cyclopent-2-en-1-yl]methanol and 4-amino-1-[(2R,5S)-2-(hydroxymethyl)-1,3-oxathiolan-5-yl]pyrimidin-2-one and 1-[(2R,4S,5S)-4-azido-5-(hydroxymethyl)oxolan-2-yl]-5-methylpyrimidine-2,4-dione. In a preferred embodiment, the antiviral active pharmaceutical ingredient is a combination of abacavir and zidovudine and lamivudine.

In a preferred embodiment, the antiviral active pharmaceutical ingredient is a combination of [(1S,4R)-4-[2-amino-6-(cyclopropylamino)purin-9-yl]cyclopent-2-en-1-yl]methanol and 4-amino-1-[(2R,5S)-2-(hydroxymethyl)-1,3-oxathiolan-5-yl]pyrimidin-2-one. In a preferred embodiment, the antiviral active pharmaceutical ingredient is a combination of abacavir and lamivudine. The preparation of this combination is described in U.S. Pat. Nos. 5,905,082, 6,294,540, and 6,417,191, the disclosures of which are incorporated by reference herein.

In a preferred embodiment, the antiviral active pharmaceutical ingredient is a combination of (4R,12aS)—N-[(2,4-difluorophenyl)methyl]-7-hydroxy-4-methyl-6,8-dioxo-3,4,12,12a-tetrahydro-2H-pyrido[5,6]pyrazino[2,6-b][1,3]oxazine-9-carboxamide and [(1S,4R)-4-[2-amino-6-(cyclopropylamino)purin-9-yl]cyclopent-2-en-1-yl]methanol and 4-amino-1-[(2R,5S)-2-(hydroxymethyl)-1,3-oxathiolan-5-yl]pyrimidin-2-one. In a preferred embodiment, the antiviral active pharmaceutical ingredient is a combination of abacavir and dolutegravir and lamivudine. The preparation of this combination is described in U.S. Pat. Nos. 5,905,082, 6,294,540, 8,129,385, 6,417,191, and 5,905,082, the disclosures of which are incorporated by reference herein.

In a preferred embodiment, the antiviral active pharmaceutical ingredient is a combination of methyl N-[(2S)-1-[2-[(2S,3S)-2-hydroxy-3-[[(2S)-2-(methoxycarbonylamino)-3,3-dimethylbutanoyl]amino]-4-phenylbutyl]-2-[(4-pyridin-2-ylphenyl)methyl]hydrazinyl]-3,3-dimethyl-1-oxobutan-2-yl]carbamate and 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl]amino]-1,6-diphenylhexan-2-yl]carbamate. In a preferred embodiment, the antiviral active pharmaceutical ingredient is a combination of atazanavir and cobicistat.

In a preferred embodiment, the antiviral active pharmaceutical ingredient is a combination of 4-amino-5-fluoro-1-[(2R,5S)-2-(hydroxymethyl)-1,3-oxathiolan-5-yl]pyrimidin-2-one and [[(2R)-1-(6-aminopurin-9-yl)propan-2-yl]oxymethyl-(propan-2-yloxycarbonyloxymethoxy)phosphoryl]oxymethyl propan-2-yl carbonate and (E)-but-2-enedioic acid and (4S)-6-chloro-4-(2-cyclopropylethynyl)-4-(trifluoromethyl)-1H-3,1-benzoxazin-2-one. In a preferred embodiment, the antiviral active pharmaceutical ingredient is a combination of efavirenz and emtricitabine and tenofovir disoproxil fumarate.

In a preferred embodiment, the antiviral active pharmaceutical ingredient is a combination of 4-amino-1-[(2R,5S)-2-(hydroxymethyl)-1,3-oxathiolan-5-yl]pyrimidin-2-one and 1-[(2R,4S,5S)-4-azido-5-(hydroxymethyl)oxolan-2-yl]-5-methylpyrimidine-2,4-dione. In a preferred embodiment, the antiviral active pharmaceutical ingredient is a combination of lamivudine and zidovudine.

In a preferred embodiment, the antiviral active pharmaceutical ingredient is a combination of [(3aS,4R,6aR)-2,3,3a,4,5,6a-hexahydrofuro[2,3-b]furan-4-yl] N-[(2S,3R)-4-[(4-aminophenyl)sulfonyl-(2-methylpropyl)amino]-3-hydroxy-1-phenylbutan-2-yl]carbamate and 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl]amino]-1,6-diphenylhexan-2-yl]carbamate. In a preferred embodiment, the antiviral active pharmaceutical ingredient is a combination of cobicistat and darunavir.

In a preferred embodiment, the antiviral active pharmaceutical ingredient is a combination of 4-amino-5-fluoro-1-[(2R,5S)-2-(hydroxymethyl)-1,3-oxathiolan-5-yl]pyrimidin-2-one and [[(2R)-1-(6-aminopurin-9-yl)propan-2-yl]oxymethyl-(propan-2-yloxycarbonyloxymethoxy)phosphoryl]oxymethyl propan-2-yl carbonate and (E)-but-2-enedioic acid and 6-[(3-chloro-2-fluorophenyl)methyl]-1-[(2S)-1-hydroxy-3-methylbutan-2-yl]-7-methoxy-4-oxoquinoline-3-carboxylic acid and 1,3-thiazol-5-ylmethyl N-[(2R,5R)-5-[[(2S)-2-[[methyl-[(2-propan-2-yl-1,3-thiazol-4-yl)methyl]carbamoyl]amino]-4-morpholin-4-ylbutanoyl] amino]-1,6-diphenylhexan-2-yl]carbamate. In a preferred embodiment, the antiviral active pharmaceutical ingredient is a combination of elvitegravir and cobicistat and emtricitabine and tenofovir disoproxil. The preparation of this combination is described in U.S. Pat. Nos. 5,814,639, 5,914,331, 5,922,695, 5,935,946, 5,977,089, 6,043,230, 6,642,245, 6,703,396, 7,176,220, 7,635,704, 8,148,374, 8,592,397, 8,633,219, and 8,716,264, the disclosures of which are incorporated by reference herein.

In a preferred embodiment, the antiviral active pharmaceutical ingredient is a combination of 4-amino-5-fluoro-1-[(2R,5S)-2-(hydroxymethyl)-1,3-oxathiolan-5-yl]pyrimidin-2-one and [[(2R)-1-(6-aminopurin-9-yl)propan-2-yl]oxymethyl-(propan-2-yloxycarbonyloxymethoxy)phosphoryl]oxymethyl propan-2-yl carbonate and (E)-but-2-enedioic acid and 4-[[4-[4-[(E)-2-cyanoethenyl]-2,6-dimethylanilino]pyrimidin-2-yl]amino] benzonitrile; hydrochloride. In a preferred embodiment, the antiviral active pharmaceutical ingredient is a combination of emtricitabine and rilpivirine and enofovir disoproxil. The preparation of this combination is described in U.S. Pat. Nos. 5,814,639, 5,914,331, 5,922,695, 5,935,946, 5,977,089, 6,043,230, 6,642,245, 6,703,396, 6,838,464, 7,067,522, 7,125,879, 8,080,551, 8,101,629, 8,592,397, 8,716,264, 8,841,310, the disclosures of which are incorporated by reference herein.

In a preferred embodiment, the antiviral active pharmaceutical ingredient is a combination of 4-amino-1-[(2R,5 S)-2-(hydroxymethyl)-1,3-oxathiolan-5-yl]pyrimidin-2-one and N-[2-[4-[(4-fluorophenyl)methylcarbamoyl]-5-hydroxy-1-methyl-6-oxopyrimidin-2-yl]propan-2-yl]-5-methyl-1,3,4-oxadiazole-2-carboxamide. In a preferred embodiment, the antiviral active pharmaceutical ingredient is a combination of lamivudine and raltegravir.

In a preferred embodiment, the antiviral active pharmaceutical ingredient is a combination of (2S)—N-[(2S,4S,5S)-5-[[2-(2,6-dimethylphenoxy)acetyl]amino]-4-hydroxy-1,6-diphenylhexan-2-yl]-3-methyl-2-(2-oxo-1,3-diazinan-1-yl)butanamide and 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate. In a preferred embodiment, the antiviral active pharmaceutical ingredient is a combination of lopinavir and ritonavir.

In a preferred embodiment, the antiviral active pharmaceutical ingredient may be any individual or combination of compounds selected from Table 1 below.

TABLE 1
Antiviral compounds and indicated viral infections.

| Antiviral | Indication | Chemical name (IUPAC) | U.S. Pat. Nos. |
|---|---|---|---|
| Abacavir | HIV | [(1S,4R)-4-[2-amino-6-(cyclopropylamino) | 5,089,500; 6,294,540; |
| | | purin-9-yl]cyclopent-2-en-1-yl]methanol | 6,641,843 |
| Aciclovir, | herpes simplex, | 2-amino-9-(2-hydroxyethoxymethyl)-3H- | 4,146,715; 8,791,127; |
| acyclovir | varicella zoster, | purin-6-one | 8,592,434; 8,747,896 |
| | chickenpox, shingles | | |
| Adefovir | hepatitis B, | 2-(6-aminopurin-9-yl) | 5,663,159; 6,451,340 |
| | herpes simplex | ethoxymethylphosphonic acid | |
| Adefovir | hepatitis B, | [2-(6-aminopurin-9-yl)ethoxymethyl-(2,2- | 5,663,159; 6,451,340 |
| dipivoxil | herpes simplex | dimethylpropanoyloxymethoxy)phosphoryl] | |
| | | oxymethyl 2,2-dimethylpropanoate | |
| Amantadine | influenza A | adamantan-1-amine | 3,152,180 |
| Amprenavir | HIV | [(3S)-oxolan-3-yl] N-[(2S,3R)-4-[(4- | 5,585,397; 6,730,679 |
| | | aminophenyl)sulfonyl-(2- | |
| | | methylpropyl)amino]-3-hydroxy-1- | |
| | | phenylbutan-2-yl]carbamate | |
| Atazanavir | HIV | methyl N-[(2S)-1-[2-[(2S,3S)-2-hydroxy- | 5,849,911; 6,087,383 |
| | | 3-[[(2S)-2-(methoxycarbonylamino)-3,3- | |
| | | dimethylbutanoyl]amino]-4-phenylbutyl]- | |
| | | 2-[(4-pyridin-2-ylphenyl)methyl] | |
| | | hydrazinyl]-3,3-dimethyl-1-oxobutan-2- | |
| | | yl]carbamate | |
| Boceprevir | hepatitis C | (1R,2S,5S)-N-(4-amino-1-cyclobutyl-3,4- | 7,772,178; 8,119,602; |
| | | dioxobutan-2-yl)-3-[(2S)-2-(tert- | RE43,298 |
| | | butylcarbamoylamino)-3,3- | |
| | | dimethylbutanoyl]-6,6-dimethyl-3- | |
| | | azabicyclo[3.1.0]hexane-2-carboxamide | |
| Cidofovir | cytomegalovirus | [(2S)-1-(4-amino-2-oxopyrimidin-1-yl)-3- | 5,142,051 |
| | (CMV) retinitis | hydroxypropan-2-yl]oxymethylphosphonic | |
| | in people with | acid | |
| | AIDS, smallpox, | | |
| | BK virus | | |
| Cobicistat | HIV | 1,3-thiazol-5-ylmethyl N-[(2R,5R)-5- | 7,939,553 |
| | | [[(2S)-2-[[methyl-[(2-propan-2-yl-1,3- | |
| | | thiazol-4-yl)methyl]carbamoyl]amino]-4- | |
| | | morpholin-4-ylbutanoyl]amino]-1,6- | |
| | | diphenylhexan-2-yl]carbamate | |
| Dolutegravir | HIV | (4R,12aS)-N-[(2,4-difluorophenyl) | 8,129,385 |
| | | methyl]-7-hydroxy-4-methyl-6,8-dioxo- | |
| | | 3,4,12,12a-tetrahydro-2H-pyrido[5,6] | |
| | | pyrazino[2,6-b][1,3]oxazine-9- | |
| | | carboxamide | |
| Darunavir | HIV | [(3aS,4R,6aR)-2,3,3a,4,5,6a- | 6,248,775; 6,335,460; |
| | | hexahydrofuro[2,3-b]furan-4-yl]N- | 7,700,645; 5843946; |
| | | [(2S,3R)-4-[(4-aminophenyl)sulfonyl-(2- | |
| | | methylpropyl)amino]-3-hydroxy-1- | 6037157; 6703403; |
| | | phenylbutan-2-yl]carbamate | 7470506; 8518987; |
| | | | 8597876; RE42889; |
| | | | RE43596; RE43802 |
| Delavirdine | HIV | N-[2-[4-[3-(propan-2-ylamino)pyridin-2- | 5,563,142; 6,177,101 |
| | | yl]piperazine-1-carbonyl]-1H-indol-5- | |
| | | yl]methanesulfonamide | |
| Didanosine | HIV | 9-[(2R,5S)-5-(hydroxymethyl)oxolan-2- | 5,880,106 |
| | | yl]-3H-purin-6-one | |
| Docosanol | herpes simplex | docosan-1-ol | 4,874,794; 5,534,554 |
| Edoxudine, | herpes simplex | 5-ethyl-1-[(2R,4S,5R)-4-hydroxy-5- | 4,267,171 |
| edoxudin | | (hydroxymethyl)oxolan-2-yl]pyrimidine- | |
| | | 2,4-dione | |
| Efavirenz | HIV | (4S)-6-chloro-4-(2-cyclopropylethynyl)- | 5,811,423; 6,238,695; |
| | | 4-(trifluoromethyl)-1H-3,1-benzoxazin- | 5,519,021; 5,663,169; |
| | | 2-one | 6,555,133; 6,639,071; |
| | | | 6,939,964 |
| Elvitegravir | HIV | 6-[(3-chloro-2-fluorophenyl)methyl]-1- | 7,635,704; 7,176,220 |
| | | [(2S)-1-hydroxy-3-methylbutan-2-yl]-7- | |
| | | methoxy-4-oxoquinoline-3-carboxylic acid | |
| Emtricitabine | HIV, hepatitis B | 4-amino-5-fluoro-1-[(2R,5S)-2- | 6,703,396; 7,402,588; |
| | | (hydroxymethyl)-1,3-oxathiolan-5- | 5,814,639; 5,914,331; |
| | | yl]pyrimidin-2-one | 6,642,245 |
| Enfuvirtide | HIV | Ac-Tyr-Thr-Ser-Leu-Ile-His-Ser-Leu-Ile- | 5,464,933; 6,475,491; |
| | | Glu-Glu-Ser-Gln-Asn-Gln-Gln-Glu-Lys-Asn- | 6,133,418 |
| | | Glu-Gln-Glu-Leu-Leu-Glu-Leu-Asp-Lys-Trp- | |
| | | Ala-Ser-Leu-Trp-Asn-Trp-Phe-NH₂ | |
| Entecavir | hepatitis B | 2-amino-9-[(1S,3R,4S)-4-hydroxy-3- | 5,206,244 |
| | | (hydroxymethyl)-2- | |
| | | methylidenecyclopentyl]-3H-purin-6-one | |
| Famciclovir | herpes zoster | [2-(acetyloxymethyl)-4-(2-aminopurin-9- | 5,246,937; 5,840,763; |
| | (shingles) | yl)butyl] acetate | 5,866,581; 5,916,893; |
| | | | 6,124,304 |
| Fomivirsen | cytomegalovirus | 5′-GCG TTT GCT CTT CTT CTT GCG-3′ | 4,689,320; 5,264,423; |
| | retinitis | | 5,276,019; 5,442,049; |
| | (CMV) in | | 5,595,978 |
| | immunocompromised | | |
| | patients, including | | |
| | those with AIDS | | |
| Fosamprenavir | HIV | [(3S)-oxolan-3-yl] N-[(2S,3R)-4-[(4- | 6,436,989; 6,514,953 |
| | | aminophenyl)sulfonyl-(2-methylpropyl) | |
| | | amino]-1-phenyl-3-phosphonooxybutan-2- | |
| | | yl]carbamate | |
| Foscarnet | herpes viruses | phosphonoformic acid | 5,072,032 |
| | (i.e. drug-resistant | | |
| | cytomegalovirus | | |
| | (CMV), herpes | | |
| | simplex viruses | | |
| | types 1/2 (HSV-1 | | |
| | & HSV-2)); CMV | | |
| | retinitis | | |
| Ganciclovir | herpes simplex virus | 2-amino-9-(1,3-dihydroxypropan-2- | 5,378,475 |
| | epithelial | yloxymethyl)-3H-purin-6-one | |
| | keratitis, | | |
| | human herpesvirus | | |
| | 6 infections, | | |
| | acute CMV colitis in | | |
| | HIV/AIDS & CMV | | |
| | pneumonitis in | | |
| | immunosuppressed | | |
| | patients, | | |
| | sight-threatening | | |
| | CMV retinitis in | | |
| | immunocompromised | | |
| | people, | | |
| | CMV pneumonitis | | |
| | in bone marrow | | |
| | transplant | | |
| | recipients, | | |
| | prevention of | | |
| | CMV disease in bone | | |
| | marrow & solid organ | | |
| | transplant | | |
| | recipients, | | |
| | CMV retinitis in | | |
| | people with AIDS | | |
| Ibacitabine | | 4-amino-1-[(2R,4S,5R)-4-hydroxy-5- | 4,845,081 |
| | | (hydroxymethyl)oxolan-2-yl]-5- | |
| | | iodopyrimidin-2-one | |
| Idoxuridine | herpes simplex | 1-[(2R,4S,5R)-4-hydroxy-5- | 5,468,853 |
| | keratitis | (hydroxymethyl)oxolan-2-yl]-5- | |
| | | iodopyrimidine-2,4-dione | |
| Imiquimod | skin cancers (basal | 1-(2-methylpropyl)imidazo[4,5- | 4,689,338; 7,696,159; |
| | cell carcinoma, | c]quinolin-4-amine | 8,236,816; 8,222,270; |
| | Bowen's disease, | | 8,299,109; 8,598,196 |
| | superficial | | |
| | squamous cell | | |
| | carcinoma, some | | |
| | superficial | | |
| | malignant melanomas, | | |
| | and actinic | | |
| | keratosis); genital | | |
| | warts (condylomata | | |
| | acuminata) | | |
| Indinavir | HIV | (2S)-1-[(2S,4R)-4-benzyl-2-hydroxy-5- | 5,413,999; 6,689,761; |
| | | [[(1S,2R)-2-hydroxy-2,3-dihydro-1H- | 6,645,961 |
| | | inden-l-yl]amino]-5-oxopentyl]-N-tert- | |
| | | butyl-4-(pyridin-3-ylmethyl)piperazine- | |
| | | 2-carboxamide | |
| Lamivudine | chronic hepatitis | 4-amino-1-[(2R,5S)-2-(hydroxymethyl)- | 6,004,968; 7,119,202; |
| | B, HIV | 1,3-oxathiolan-5-yl]pyrimidin-2-one | 5,905,082; RE39155 |
| Lopinavir | HIV | (2S)-N-[(2S,4S,5S)-5-[[2-(2,6- | 5,914,332 |
| | | dimethylphenoxy)acetyl]amino]-4-hydroxy- | |
| | | 1,6-diphenylhexan-2-yl]-3-methyl-2-(2- | |
| | | oxo-1,3-diazinan-1-yl)butanamide | |
| Loviride | HIV | 2-(2-acetyl-5-methylanilino)-2-(2,6- | 5,556,886 |
| | | dichlorophenyl)acetamide | |
| Maraviroc | HIV | 4,4-difluoro-N-[(1S)-3-[(1S,5R)-3-(3- | 6,586,430; 7,368,460; |
| | | methyl-5-propan-2-yl-1,2,4-triazol-4- | 6,667,314; 7,576,097 |
| | | yl)-8-azabicyclo[3.2.1]octan-8-yl]-1- | |
| | | phenylpropyl]cyclohexane-1-carboxamide | |
| Maribavir | CMV in immuno- | (2S,3S,4R,5S)-2-[5,6-dichloro-2-(propan- | 6,469,160; 6,204,249; |
| | compromised | 2-ylamino)benzimidazol-1-yl]-5- | 6,617,315; 6,482,939; |
| | patients | (hydroxymethyl)oxolane-3,4-diol | 6,307,043; 7,858,773; |
| | | | 2010/0179101; |
| | | | 2011/0118203 |
| Moroxydine | potential | N-(diaminomethylidene)morpholine-4- | 4,656,291; 4,372,954; |
| | applications | carboximidamide | 4,089,957 |
| | against RNA | | |
| | and DNA viruses | | |
| Methisazone | smallpox | [(Z)-(1-methyl-2-oxoindol-3- | |
| | | ylidene)amino]thiourea | |
| Nelfinavir | HIV | (3S,4aS,8aS)-N-tert-butyl-2-[(2R,3R)-2- | 5,484,926; 5,952,343; |
| | | hydroxy-3-[(3-hydroxy-2-methylbenzoyl) | 6,162,812 |
| | | amino]-4-phenylsulfanylbutyl]- | |
| | | 3,4,4a,5,6,7,8,8a-octahydro-1H- | |
| | | isoquinoline-3-carboxamide | |
| Nevirapine | HIV | 11-cyclopropyl-4-methyl-5H-dipyrido | 5,366,972; 8,460,704 |
| | | [2,3-e:2′,3′-f][1,4]diazepin-6-one | |
| Oseltamivir | influenza A, | ethyl (3R,4R,5S)-4-acetamido-5-amino-3- | 5,763,483; 5,866,601; |
| | influenza B | pentan-3-yloxycyclohexene-1-carboxylate | 5,952,375 |
| Penciclovir | herpesvirus | 2-amino-9-[4-hydroxy-3-(hydroxymethyl) | 5,075,445; 6,579,981; |
| | | butyl]-3H-purin-6-one | 5,840,763; 6,124,304; |
| | | | 5,916,893; 6,469,015; |
| | | | 5,866,581 |
| Peramivir | influenza, swine flu | (1S,2S,3S,4R)-3-[(1S)-1-acetamido-2- | 2011/0065795; |
| | | ethylbutyl]-4-(diaminomethylideneamino)- | 2013/0331604 |
| | | 2-hydroxycyclopentane-1-carboxylic acid | |
| Pleconaril | picornavirus | 3-[3,5-dimethyl-4-[3-(3-methyl-1,2- | 5,464,848; 7,060,708 |
| | respiratory | oxazol-5-yl)propoxy]phenyl]-5- | |
| | infections | (trifluoromethyl)-1,2,4-oxadiazole | |
| | (i.e. enterovirus, | | |
| | rhinovirus) | | |
| Podophyllotoxin | human papillomavirus | (5R,5aR,8aR,9R)-5-hydroxy-9-(3,4,5- | 5,057,616; 5,315,016 |
| | (HPV) | trimethoxyphenyl)-5a,6,8a,9-tetrahydro- | |
| | | 5H-[2]benzofuro[5,6-f][1,3]benzodioxol- | |
| | | 8-one | |
| Raltegravir | HIV | N-[2-[4-[(4-fluorophenyl) | 7,169,780; 7,217,713; |
| | | methylcarbamoyl]-5-hydroxy-1-methyl-6- | 7,754,731; 7,435,734 |
| | | oxopyrimidin-2-yl]propan-2-yl]-5-methyl- | |
| | | 1,3,4-oxadiazole-2-carboxamide | |
| Ribavirin | hepatitis C, viral | 1-[(2R,3R,4S,5R)-3,4-dihydroxy-5- | 6,177,074; 6,790,837; |
| | hemorrhagic fevers | (hydroxymethyl)oxolan-2-yl]-1,2,4- | 6,150,337; 6,172,046; |
| | | triazole-3-carboxamide | 6,461,605; 6,472,373; |
| | | | 6,524,570 |
| Rilpivirine | HIV | 4-[[4-[4-[(E)-2-cyanoethenyl]-2,6- | 6,838,464; 7,067,522; |
| | | dimethylanilino]pyrimidin-2- | 7,125,879; 7,638,522; |
| | | yl]amino]benzonitrile | 8,080,551; 8,101,629 |
| Rimantadine | influenza A | 1-(1-adamantyl)ethanamine | 4,027,035; 4,551,552; |
| | | | 3,352,912 |
| Ritonavir | HIV | 1,3-thiazol-5-ylmethyl N-[(2S,3S,5S)-3- | 5,541,206; 7,364,752; |
| | | hydroxy-5-[[(2S)-3-methyl-2-[[methyl- | 5,484,801; 5,635,523; |
| | | [(2-propan-2-yl-1,3-thiazol-4-yl)methyl] | 5,648,497; 5,674,882; |
| | | carbamoyl]amino]butanoyl]amino]-1,6- | 5,948,436; 6,037,157; |
| | | diphenylhexan-2-yl]carbamate | 6,232,333; 6,703,403; |
| | | | 7,141,593; 7,148,359; |
| | | | 7,432,294; 8,268,349; |
| | | | 8,399,015; 8,470,347; |
| | | | 8,691,878 |
| Saquinavir | HIV | (2S)-N-[(2S,3R)-4-[(3S,4aS,8aS)-3- | 5,196,438; 6,352,717 |
| | | (tert-butylcarbamoyl)-3,4,4a,5,6,7,8,8a- | |
| | | octahydro-1H-isoquinolin-2-yl]-3- | |
| | | hydroxy-1-phenylbutan-2-yl]-2- | |
| | | (quinoline-2-carbonylamino)butanediamide | |
| Sofosbuvir | chronic hepatitis C | propan-2-yl (2S)-2-[[[(2R,3R,4R,5R)-5- | 2010/0298257 |
| | | (2,4-dioxopyrimidin-1-yl)-4-fluoro-3- | |
| | | hydroxy-4-methyloxolan-2-yl]methoxy- | |
| | | phenoxyphosphoryl]amino]propanoate | |
| Stavudine | HIV | 1-[(2R,5S)-5-(hydroxymethyl)-2,5- | 7,135,465; 8,026,356; |
| | | dihydrofuran-2-yl]-5-methylpyrimidine- | 5,130,421 |
| | | 2,4-dione | |
| Telaprevir | hepatitis C | (3S,3aS,6aR)-2-[(2S)-2-[[(2S)-2- | 7,820,671; 8,529,882; |
| | | cyclohexyl-2-(pyrazine-2-carbonylamino) | 8,431,615 |
| | | acetyl]amino]-3,3-dimethylbutanoyl]-N- | |
| | | [(3S)-1-(cyclopropylamino)-1,2- | |
| | | dioxohexan-3-yl]-3,3a,4,5,6,6a- | |
| | | hexahydro-1H-cyclopenta[c]pyrrole-3- | |
| | | carboxamide | |
| Tenofovir | HIV-1, chronic | [(2R)-1-(6-aminopurin-9-yl)propan-2- | 5,922,695; 8,049,009 |
| | hepatitis B | yl]oxymethylphosphonic acid | |
| Tenofovir | HIV-1, chronic | [[(2R)-1-(6-aminopurin-9-yl)propan-2- | 5,935,946; 5,922,695; |
| disoproxil | hepatitis B | yl]oxymethyl-(propan-2- | 5,977,089; 6,043,230; |
| | | yloxycarbonyloxymethoxy)phosphoryl] | 6,069,249 |
| | | oxymethyl propan-2-yl carbonate | |
| Tipranavir | HIV | N-[3-[(1R)-1-[(2R)-4-hydroxy-6-oxo-2- | 6,147,095; 6,169,181; |
| | | (2-phenylethyl)-2-propyl-3H-pyran-5- | 7,002,017; 5,852,195; |
| | | yl]propyl]phenyl]-5-(trifluoromethyl) | 6,231,887 |
| | | pyridine-2-sulfonamide | |
| Trifluridine | herpesvirus | 1-[(2R,4S,5R)-4-hydroxy-5- | 3,201,387; 3,531,464 |
| | | (hydroxymethyl)oxolan-2-yl]-5- | |
| | | (trifluoromethyl)pyrimidine-2,4-dione | |
| Tromantadine | herpes simplex | N-(1-adamantyl)-2-[2-(dimethylamino) | 2013/0072553 |
| | virus | ethoxy]acetamide | |
| Valaciclovir, | herpes simplex, | 2-[(2-amino-6-oxo-3H-purin-9-yl) | 4,957,924; 5,879,706; |
| valacyclovir | herpes zoster | methoxy]ethyl (2S)-2-amino-3- | 6,849,737; 6,107,302 |
| | (shingles), herpes B | methylbutanoate | |
| Valganciclovir | cytomegalovirus | [2-[(2-amino-6-oxo-3H-purin-9- | 6,083,953; |
| | infections | yl)methoxy]-3-hydroxypropyl](2S)- | 2007/0292499 |
| | | 2-amino-3-methylbutanoate | |
| Vicriviroc | HIV-1 | (4,6-dimethylpyrimidin-5-yl)-[4-[(3S)- | 7,534,884; 6,391,865; |
| | | 4-[(1R)-2-methoxy-1-[4-(trifluoromethyl) | 6,943,251; 6,689,765 |
| | | phenyl]ethyl]-3-methylpiperazin-1-yl]- | |
| | | 4-methylpiperidin-1-yl]methanone | |
| Vidarabine | herpes simplex virus, | (2R,3S,4S,5R)-2-(6-aminopurin-9-yl)-5- | 3,703,507; 4,123,609; |
| | varicella zoster | (hydroxymethyl)oxolane-3,4-diol | 5,506,352 |
| | virus, herpes | | |
| | viruses, poxviruses, | | |
| | rhabdoviruses, | | |
| | hepadnaviruses, RNA | | |
| | tumour viruses | | |
| Viramidine, | some DNA and RNA | 1-[(2R,3R,4S,5R)-3,4-dihydroxy-5- | 4,925,930; 7,638,496 |
| taribavirin | viruses; viral | (hydroxymethyl)oxolan-2-yl]-1,2,4- | |
| | hepatitis syndromes | triazole-3-carboximidamide | |
| | where ribavirin | | |
| | is active (hepatitis | | |
| | C, hepatitis B, | | |
| | yellow fever) | | |
| Zalcitabine | HIV | 4-amino-1-[(2R,5S)-5-(hydroxymethyl) | 4,879,277; 5,028,595 |
| | | oxolan-2-yl]pyrimidin-2-one | |
| Zanamivir | influenza A, | (2R,3R,4S)-3-acetamido-4- | 5,360,817; 6,294,572. |
| | influenza B | (diaminomethylideneamino)-2-[(1R,2R)- | 2011/0257418; |
| | | 1,2,3-trihydroxypropyl]-3,4-dihydro-2H- | 5,648,379 |
| | | pyran-6-carboxylic acid | |
| Zidovudine | HIV | 1-[(2R,4S,5S)-4-azido-5-(hydroxymethyl) | 4,724,232 |
| | | oxolan-2-yl]-5-methylpyrimidine-2,4- | |
| | | dione | |

### Anti-Inflammatory Active Pharmaceutical Ingredients

The anti-inflammatory active pharmaceutical ingredients of the invention may include a steroid or a non-steroidal anti-inflammatory drug (NSAID). In an embodiment, the invention includes an anti-inflammatory active pharmaceutical ingredient comprising a steroid, such as dexamethasone, dexamethasone alcohol, dexamethasone sodium phosphate, fluromethalone acetate, fluromethalone alcohol, loteprednol etabonate, medrysone, prednisolone acetate, prednisolone sodium phosphate, prednisone, difluprednate, rimexolone, hydrocortisone, hydrocortisone acetate, lodoxamide tromethamine, salts, derivatives, esters, and prodrugs thereof, and any combinations thereof. These steroids are commerically available and are well-known to those of skill in the art.

In an embodiment, the steroid is a compound selected from the structures disclosed in U.S. Pat. No. 8,771,722 B2, and U.S. Patent Application Publication Nos. US 2013/0245253 A1 and US 2014/0256651 A1.

In a preferred embodiment, the steroid is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one. In a preferred embodiment, the steroid is dexamethasone, or a pharmaceutically acceptable salt, solvate, hydrate, cocrystal, or prodrug thereof. The preparation of this compound is described in U.S. Pat. Nos. 2,852,511; 3,007,923; 6,899,717; 6,726,918; 7,033,605; 8,088,407; 8,506,987; 8,043,628; 8,063,031; 8,034,366; 7,767,223; 8,034,370, the disclosures of which are incorporated by reference herein. In an embodiment, the steroid is selected from the compounds described in U.S. Pat. Nos. 2,852,511; 3,007,923; 6,899,717; 6,726,918; 7,033,605; 8,088,407; 8,506,987; 8,043,628; 8,063,031; 8,034,366; 7,767,223; 8,034,370, the disclosures of which are incorporated by reference herein.

In a preferred embodiment, the steroid is disodium; [2-[(8S,9R,10S,11S,13S,14S,16R,17R)-9-fluoro-11,17-dihydroxy-10,13,16-trimethyl-3-oxo-6,7,8,11,12,14,15,16-octahydrocyclopenta[a]phenanthren-17-yl]-2-oxoethyl] phosphate. In a preferred embodiment, the steroid is dexamethasone sodium phosphate, or a pharmaceutically acceptable solvate, hydrate, cocrystal, or prodrug thereof.

In a preferred embodiment, the steroid is [(6S,9R,17R)-17-acetyl-9-fluoro-11-hydroxy-6,10,13-trimethyl-3-oxo-6,7,8,11,12,14,15,16-octahydrocyclopenta[a]phenanthren-17-yl] acetate. In a preferred embodiment, the steroid is fluromethalone acetate, or a pharmaceutically acceptable salt, solvate, hydrate, cocrystal, or prodrug thereof. The preparation of this compound is described in U.S. Pat. Nos. 2,867,637; 2,867,638; 2,852,511; and 3,038,914, the disclosures of which are incorporated by reference herein. In an embodiment, the steroid is selected from the compounds described in U.S. Pat. Nos. 2,867,637; 2,867,638; 2,852,511; and 3,038,914, the disclosures of which are incorporated by reference herein.

In a preferred embodiment, the steroid is chloromethyl (8S,9S,10R,11S,13S,14S,17R)-17-ethoxycarbonyloxy-11-hydroxy-10,13-dimethyl-3-oxo-7,8,9,11,12,14,15,16-octahydro-6H-cyclopenta[a]phenanthrene-17-carboxylate. In a preferred embodiment, the steroid is loteprednol etabonate, or a pharmaceutically acceptable salt, solvate, hydrate, cocrystal, or prodrug thereof. The preparation of this compound is described in U.S. Pat. Nos. 4,996,335; 5,540,930; 5,747,061; and 5,800,807, the disclosures of which are incorporated by reference herein. In an embodiment, the steroid is selected from the compounds described in U.S. Pat. Nos. 4,996,335; 5,540,930; 5,747,061; and 5,800,807, the disclosures of which are incorporated by reference herein.

In a preferred embodiment, the steroid is (6S,8S,9S,10R,11S,13S,14S,17S)-17-acetyl-11-hydroxy-6,10,13-trimethyl-1,2,6,7,8,9,11,12,14,15,16,17-dodecahydrocyclopenta[a] phenanthren-3-one. In a preferred embodiment, the steroid is medrysone, or a pharmaceutically acceptable salt, solvate, hydrate, cocrystal, or prodrug thereof.

In a preferred embodiment, the steroid is [2-[(8S,9S,10R,11S,13S,14S,17R)-11,17-dihydroxy-10,13-dimethyl-3-oxo-7,8,9,11,12,14,15,16-octahydro-6H-cyclopenta[a] phenanthren-17-yl]-2-oxoethyl] acetate. In a preferred embodiment, the steroid is prednisolone acetate, or a pharmaceutically acceptable salt, solvate, hydrate, cocrystal, or prodrug thereof. The preparation of this compound is described in U.S. Pat. Nos. 6,071,523; 6,399,079; 7,799,331; 5,881,926; and 6,656,482, the disclosures of which are incorporated by reference herein. In an embodiment, the steroid is selected from the compounds described in U.S. Pat. Nos. 6,071,523; 6,399,079; 7,799,331; 5,881,926; and 6,656,482, the disclosures of which are incorporated by reference herein.

In a preferred embodiment, the steroid is disodium [2-[(10R,11S,13S,17R)-11,17-dihydroxy-10,13-dimethyl-3-oxo-7,8,9,11,12,14,15,16-octahydro-6H-cyclopenta[a]phenanthren-17-yl]-2-oxoethyl] phosphate. In a preferred embodiment, the steroid is prednisolone sodium phosphate, or a pharmaceutically acceptable solvate, hydrate, cocrystal, or prodrug thereof.

In a preferred embodiment, the steroid is [(6S,8S,9R,10S,11S,13S,14S,17R)-17-(2-acetyloxyacetyl)-6,9-difluoro-11-hydroxy-10,13-dimethyl-3-oxo-6,7,8,11,12,14,15,16-octahydrocyclopenta[a]phenanthren-17-yl] butanoate. In a preferred embodiment, the steroid is difluprednate, or a pharmaceutically acceptable salt, solvate, hydrate, cocrystal, or prodrug thereof. The preparation of this compound is described in U.S. Pat. Nos. 3,780,177 and 6,114,319, the disclosures of which are incorporated by reference herein. In an embodiment, the steroid is selected from the compounds described in U.S. Pat. Nos. 3,780,177 and 6,114,319, the disclosures of which are incorporated by reference herein.

In a preferred embodiment, the steroid is (8S,9S,10R,11S,13S,14S,16R,17S)-11-hydroxy-10,13,16,17-tetramethyl-17-propanoyl-7,8,9,11,12,14,15,16-octahydro-6H-cyclopenta[a]phenanthren-3-one. In a preferred embodiment, the steroid is rimexolone, or a pharmaceutically acceptable salt, solvate, hydrate, cocrystal, or prodrug thereof. The preparation of this compound is described in U.S. Pat. No. 7,432,261, the disclosure of which is incorporated by reference herein. In an embodiment, the steroid is selected from the compounds described in U.S. Pat. No. 7,432,261, the disclosure of which is incorporated by reference herein.

In a preferred embodiment, the steroid is (8S,9S,10R,11S,13S,14S,17R)-11,17-dihydroxy-17-(2-hydroxyacetyl)-10,13-dimethyl-2,6,7,8,9,11,12,14,15,16-decahydro-1H-cyclopenta[a]phenanthren-3-one. In a preferred embodiment, the steroid is hydrocortisone, or a pharmaceutically acceptable salt, solvate, hydrate, cocrystal, or prodrug thereof. The preparation of this compound is described in U.S. Pat. Nos. 5,635,497; 7,378,405; and 4,264,584, the disclosures of which are incorporated by reference herein. In an embodiment, the steroid is selected from the compounds described in U.S. Pat. Nos. 5,635,497; 7,378,405; and 4,264,584, the disclosures of which are incorporated by reference herein.

In a preferred embodiment, the steroid is [2-[(8S,9S,10R,11S,13S,14S,17R)-11,17-dihydroxy-10,13-dimethyl-3-oxo-2,6,7,8,9,11,12,14,15,16-decahydro-1H-cyclopenta[a] phenanthren-17-yl]-2-oxoethyl] acetate. In a preferred embodiment, the steroid is hydrocortisone acetate, or a pharmaceutically acceptable salt, solvate, hydrate, cocrystal, or prodrug thereof. The preparation of this compound is described in U.S. Pat. Nos. 2,671,750; 2,805,232; and 3,238,102, the disclosures of which are incorporated by reference herein. In an embodiment, the steroid is selected from the compounds described in U.S. Pat. Nos. 2,671,750; 2,805,232; and 3,238,102, the disclosures of which are incorporated by reference herein.

In a preferred embodiment, the steroid is 2-amino-2-(hydroxymethyl)propane-1,3-diol; 2-[2-chloro-5-cyano-3-(oxaloamino)anilino]-2-oxoacetic acid. In a preferred embodiment, the steroid is lodoxamide tromethamine, or a pharmaceutically acceptable salt, solvate, hydrate, cocrystal, or prodrug thereof. The preparation of this compound is described in U.S. Pat. No. 5,457,126, the disclosure of which is incorporated by reference herein. In an embodiment, the steroid is selected from the compounds described in U.S. Pat. No. 5,457,126, the disclosure of which is incorporated by reference herein.

In an embodiment, the invention includes an anti-inflammatory active pharmaceutical ingredient comprising an NSAID, such as flurbiprofen, ketorlac tromethamine, suprofen, celecoxib, naproxen, rofecoxib, salts, derivatives, prodrugs, and esters thereof, and any combinations thereof. These NSAIDs are commerically available and are well-known to those of skill in the art.

### Pharmaceutical Compositions

In one embodiment, the invention provides a pharmaceutical composition comprising a combination of a steroid and an antiviral active pharmaceutical ingredient. Said pharmaceutical composition typically also comprises at least one pharmaceutically acceptable excipient.

In selected embodiments, the invention provides a pharmaceutical composition comprising a combination of a steroid and an antiviral active pharmaceutical ingredient for treating viral infections.

In selected embodiments, the invention provides a pharmaceutical composition comprising a combination of a steroid and an antiviral active pharmaceutical ingredient for the treatment of a viral infection, wherein the viral infection is selected from the group consisting of a viral infection of the eye, a viral infection of the salivary glands, a viral infection of the skin, a viral infection of the liver, a viral infection of the ear, a viral infection of the brain or spine, viral meningitis infection, a viral infection of the lung, a viral infection of the nasal mucosa, and a systematic viral infection, and a viral infection of a mucous membrane.

The pharmaceutical compositions are typically formulated to provide a therapeutically effective amount of a combination of a steroid and an antiviral active pharmaceutical ingredient as the active ingredients, or a pharmaceutically acceptable salt, ester, prodrug, solvate, or hydrate thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The pharmaceutical compositions are administered as a combination of a steroid and an antiviral active pharmaceutical ingredient. Where desired, other agent(s) may be mixed into a preparation or both components may be formulated into separate preparations for use in combination separately or at the same time. A kit containing both components formulated into separate preparations for said use in also provided by the invention.

The weight ratio of the steroid to the antiviral active pharmaceutical ingredient in the combination is typically in the range from 1:100 to 100:1, preferably from 1:10 to 10:1, more preferably from 2.5:1 to 1:2.5, and still more preferably about 1:1.

In selected embodiments, the concentration of each of the anti-inflammatory and antiviral active pharmaceutical ingredients provided in the pharmaceutical compositions of the invention is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of each of the antiviral active pharmaceutical ingredients or steroids.

In selected embodiments, the concentration of each of the anti-inflammatory and antiviral active pharmaceutical ingredients provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of each of the antiviral active pharmaceutical ingredients or steroids.

In selected embodiments, the concentration of each of the anti-inflammatory and antiviral active pharmaceutical ingredients of the invention is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v. v/v of each of the steroids and antiviral active pharmaceutical ingredients.

In selected embodiments, the concentration of each of the anti-inflammatory and antiviral active pharmaceutical ingredients of the invention is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of each of the steroids or antiviral active pharmaceutical ingredients.

In selected embodiments, the amount of each of the anti-inflammatory and antiviral active pharmaceutical ingredients of the invention is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of each of the anti-inflammatory and antiviral active pharmaceutical ingredients of the invention is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

Each of the anti-inflammatory and antiviral active pharmaceutical ingredients according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

In an embodiment, a pharmaceutical composition retains 99% of each of the anti-inflammatory and antiviral active pharmaceutical ingredients after a period of 1 month. In an embodiment, a pharmaceutical composition retains 98% of each of the anti-inflammatory and antiviral active pharmaceutical ingredients after a period of 1 month. In an embodiment, a pharmaceutical composition retains 97% of each of the anti-inflammatory and antiviral active pharmaceutical ingredients after a period of 1 month. In an embodiment, a pharmaceutical composition retains 96% of each of the anti-inflammatory and antiviral active pharmaceutical ingredients after a period of 1 month. In an embodiment, a pharmaceutical composition retains 95% of each of the anti-inflammatory and antiviral active pharmaceutical ingredients after a period of 1 month. In an embodiment, a pharmaceutical composition retains 90% of each of the anti-inflammatory and antiviral active pharmaceutical ingredients after a period of 3 months. In an embodiment, a pharmaceutical composition retains 90% of each of the anti-inflammatory and antiviral active pharmaceutical ingredients after a period of 1 month.

In an embodiment, a pharmaceutical composition PVP-I and at least one steroid retains about 89% of its PVP-I after a period of 1 month, about 90% of its PVP-I after a period of 1 month, about 91% of its PVP-I after a period of 1 month, about 92% of its PVP-I after a period of 1 month, about 93% of its PVP-I after a period of 1 month, about 94% of its PVP-I after a period of 1 month, about 94% of its PVP-I after a period of 1 month, about 95% of its PVP-I after a period of 1 month, about 96% of its PVP-I after a period of 1 month, about 97% of its PVP-I after a period of 1 month, about 98% of its PVP-I after a period of 1 month, or about 99% of its PVP-I after a period of 1 month.

In an embodiment, a pharmaceutical composition comprising PVP-I and at least one NSAID retains about 89% of its PVP-I after a period of 1 month, about 90% of its PVP-I after a period of 1 month, about 91% of its PVP-I after a period of 1 month, about 92% of its PVP-I after a period of 1 month, about 93% of its PVP-I after a period of 1 month, about 94% of its PVP-I after a period of 1 month, about 94% of its PVP-I after a period of 1 month, about 95% of its PVP-I after a period of 1 month, about 96% of its PVP-I after a period of 1 month, about 97% of its PVP-I after a period of 1 month, about 98% of its PVP-I after a period of 1 month, or about 99% of its PVP-I after a period of 1 month.

In an embodiment, a pharmaceutical composition comprising PVP-I and at least one steroid retains about 89% of its PVP-I after a period of 3 months, about 90% of its PVP-I after a period of 3 months, about 91% of its PVP-I after a period of 3 months, about 92% of its PVP-I after a period of 3 months, about 93% of its PVP-I after a period of 3 months, about 94% of its PVP-I after a period of 3 months, about 94% of its PVP-I after a period of 3 months, about 95% of its PVP-I after a period of 3 months, about 96% of its PVP-I after a period of 3 months, about 97% of its PVP-I after a period of 3 months, about 98% of its PVP-I after a period of 3 months, or about 99% of its PVP-I after a period of 3 months.

In an embodiment, an ophthalmic composition comprising PVP-I and at least one NSAID retains about 89% of its PVP-I after a period of 3 months, about 90% of its PVP-I after a period of 3 months, about 91% of its PVP-I after a period of 3 months, about 92% of its PVP-I after a period of 3 months, about 93% of its PVP-I after a period of 3 months, about 94% of its PVP-I after a period of 3 months, about 94% of its PVP-I after a period of 3 months, about 95% of its PVP-I after a period of 3 months, about 96% of its PVP-I after a period of 3 months, about 97% of its PVP-I after a period of 3 months, about 98% of its PVP-I after a period of 3 months, or about 99% of its PVP-I after a period of 3 months.

In an embodiment, an ophthalmic composition comprising PVP-I and at least one steroid retains about 89% of its at least one steroid after a period of 1 month, about 90% of its at least one steroid after a period of 1 month, about 91% of its at least one steroid after a period of 1 month, about 92% of its at least one steroid after a period of 1 month, about 93% of its at least one steroid after a period of 1 month, about 94% of its at least one steroid after a period of 1 month, about 94% of its at least one steroid after a period of 1 month, about 95% of its at least one steroid after a period of 1 month, about 96% of its at least one steroid after a period of 1 month, about 97% of its at least one steroid after a period of 1 month, about 98% of its at least one steroid after a period of 1 month, or about 99% of its at least one steroid after a period of 1 month.

In an embodiment, an ophthalmic composition comprising PVP-I and at least one NSAID retains about 89% of its at least one NSAID after a period of 1 month, about 90% of its at least one NSAID after a period of 1 month, about 91% of its at least one NSAID after a period of 1 month, about 92% of its at least one NSAID after a period of 1 month, about 93% of its at least one NSAID after a period of 1 month, about 94% of its at least one NSAID after a period of 1 month, about 94% of its at least one NSAID after a period of 1 month, about 95% of its at least one NSAID after a period of 1 month, about 96% of its at least one NSAID after a period of 1 month, about 97% of its at least one NSAID after a period of 1 month, about 98% of its at least one NSAID after a period of 1 month, or about 99% of its at least one NSAID after a period of 1 month.

In an embodiment, an ophthalmic composition comprising PVP-I and at least one steroid retains about 89% of its at least one steroid after a period of 3 months, about 90% of its at least one steroid after a period of 3 months, about 91% of its at least one steroid after a period of 3 months, about 92% of its at least one steroid after a period of 3 months, about 93% of its at least one steroid after a period of 3 months, about 94% of its at least one steroid after a period of 3 months, about 94% of its at least one steroid after a period of 3 months, about 95% of its at least one steroid after a period of 3 months, about 96% of its at least one steroid after a period of 3 months, about 97% of its at least one steroid after a period of 3 months, about 98% of its at least one steroid after a period of 3 months, or about 99% of its at least one steroid after a period of 3 months.

In an embodiment, an ophthalmic composition comprising PVP-I and at least one NSAID retains about 89% of its at least one NSAID after a period of 3 months, about 90% of its at least one NSAID after a period of 3 months, about 91% of its at least one NSAID after a period of 3 months, about 92% of its at least one NSAID after a period of 3 months, about 93% of its at least one NSAID after a period of 3 months, about 94% of its at least one NSAID after a period of 3 months, about 94% of its at least one NSAID after a period of 3 months, about 95% of its at least one NSAID after a period of 3 months, about 96% of its at least one NSAID after a period of 3 months, about 97% of its at least one NSAID after a period of 3 months, about 98% of its at least one NSAID after a period of 3 months, or about 99% of its at least one NSAID after a period of 3 months.

Described below are non-limiting exemplary pharmaceutical compositions and methods for preparing the same.

### Pharmaceutical Compositions for Oral Administration

In selected embodiments, the invention provides a pharmaceutical composition for oral administration containing the combination of a steriod and an antiviral active pharmaceutical ingredient, and at least one pharmaceutical excipient suitable for oral administration.

In selected embodiments, the invention provides a solid pharmaceutical composition for oral administration containing: (i) an effective amount of each of a steroid and an antiviral active pharmaceutical ingredient in combination and (ii) a pharmaceutical excipient suitable for oral administration. In selected embodiments, the composition further contains (iii) an effective amount of a fourth compound.

In selected embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The invention further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the anti-inflammatory and antiviral active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an exemplary embodiment, the composition may include a solubilizer to ensure good solubilization and/or dissolution of the compound of the present invention and to minimize precipitation of the compound of the present invention. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In selected embodiments, the invention provides a pharmaceutical composition for injection containing the combination of the anti-inflammatory and antiviral active pharmaceutical ingredients and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating the combination of the anti-inflammatory and antiviral active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Ophthalmic Delivery

The compositions disclosed herein can be administered as solutions, suspensions, emulsions (dispersions), gels, creams, or ointments in a suitable ophthalmic vehicle. In any of the compositions of this disclosure for topical administration, such as topical administration to the eye, the mixtures are preferably formulated as aqueous solutions at a pH of 3.5 to 6.5. Preferentially the pH is adjusted to between 4 and 5. This pH range may be achieved by the addition of acids/bases to the solution. The ophthalmic composition may be in the form of a solution, a suspension, an emulsion, a preparation, an ointment, a cream, a gel, or a controlled-release/sustain-release vehicle. By way of a non-limiting example, the composition may be in the form of a contact lens solution, eyewash, eyedrop, and the like.

In an embodiment, an ophthalmic composition may comprise an optional co-solvent. In another embodiment, the solubility of the components of the present compositions may be enhanced by a surfactant or other appropriate co-solvent in the composition. Such co-solvents or surfactants include polysorbate-20, -60, and -80, a polyoxyethylene/polyoxypropylene surfactant (e.g., Pluronic F-68, F-84 and P-103), cyclodextrin, tyloxapol, PEG 35 Castor oil (Cremophor EL), polyoxyl 40 Stearate (Myrj 52), other agents known to those skilled in the art, or a combination thereof. Typically, such co-solvents are present at a level of from about 0.01% to about 2% by weight. In an embodiment, a co-solvent is present at a level of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In an embodiment, a composition may comprise an optional agent that can increase viscosity. As will be understood by the skilled artisan when armed with the present disclosure, it may be desirable to increase viscosity above that of a simple aqueous solution in order to increase ocular absorption of the active compound, to decrease variability in dispensing the formulation, to decrease physical separation of components of a suspension or emulsion of the formulation and/or to otherwise improve the ophthalmic formulation. Such viscosity-enhancing agents include, but are not limited to, polyvinyl alcohol, polyvinyl pyrrolidone, methyl cellulose, hydroxypropylmethyl cellulose, hydroxyethyl cellulose, carboxymethyl cellulose, hydroxypropyl cellulose, other agents known to those skilled in the art, or any combination thereof. Such agents are typically employed at a level of from about 0.01% to about 2% by weight. In an embodiment, such optional agents are present at about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In another aspect, bioadhesive agents may comprise the compositions, in order to increase the retention or residence time of the drug gradient over a biological substrate. The bioadhesive agents include, but are not limited to, polyvinylpyrrolidone (PVP), xanthan gum, locust bean gum, acacia gum, hydroxypropyl methylcellulose (HPMC), sodium alginate, pectin, gelatin, carbomer, polyvinylalcohol, gellan gum, tragacanth, acacia, and sodium carboxymethyl cellulose, as well as other agents known to those skilled in the art, or any combination thereof. In yet another embodiment, compositions of the invention may comprise viscoelastic agents such as methyl cellulose, carboxymethyl cellulose, hydroxyethyl cellulose, polyvinyl alcohol, dextran, chondroitin sulfate and salts thereof, and hyaluronic acid and salts thereof.

In an embodiment, an ophthalmic composition may further comprise one or more of (1) a penetration enhancer which enhances the penetration of povidone-iodine into the tissues of the eye (this may be a topical anesthetic) (2) a co-solvent or a nonionic surface agent—surfactant, which, for example, may be about 0.01% to 2% by weight; (3) a viscosity increasing agent, which, for example, may be about 0.01% to 2% by weight; and (4) a suitable ophthalmic vehicle.

In an embodiment, an ophthalmic composition is an aqueous solution. In an embodiment, an ophthalmic composition is a stable suspension.

### Pharmaceutical Compositions for Topical Delivery

In some embodiments, the invention provides a pharmaceutical composition for transdermal delivery containing the combination of the anti-inflammatory and antiviral active pharmaceutical ingredients and a pharmaceutical excipient suitable for transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of the combination of the anti-inflammatory and antiviral active pharmaceutical ingredients in controlled amounts, either with or without another agent.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990.

Administration of the combination of the anti-inflammatory and antiviral active pharmaceutical ingredients or pharmaceutical compositions thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. The combination of the anti-inflammatory and antiviral active pharmaceutical ingredients may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. The combination of the anti-inflammatory and antiviral active pharmaceutical ingredients may additionally be administered intravascularly from a balloon used during angioplasty. Extravascular administration of the combination of the anti-inflammatory and antiviral active pharmaceutical ingredients via the pericard or via advential application of formulations of the invention may also be performed to decrease restenosis.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include each of the anti-inflammatory and antiviral active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In selected embodiments, the anti-inflammatory and antiviral active pharmaceutical ingredients and the agent are provided as separate compositions in separate containers within the kit. In selected embodiments, the anti-inflammatory and antiviral active pharmaceutical ingredients and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

### Dosages and Dosing Regimens

The amounts of the combination of the antiviral active pharmaceutical ingredients and anti-inflammatory active pharmaceutical ingredients administered will be dependent on the mammal being treated, the severity of the disorder or condition, the rate of administration, the disposition of the compounds, and the discretion of the prescribing physician. However, an effective dosage of an antiviral active pharmaceutical ingredient or an anti-inflammatory active pharmaceutical ingredient is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day.

In some embodiments, an effective dosage of an antiviral active pharmaceutical ingredient disclosed herein, administered in combination with an anti-inflammatory active pharmaceutical ingredient, is in the range of about 1 mg to about 300 mg, about 10 mg to about 250 mg, about 20 mg to about 225 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an antiviral active pharmaceutical ingredient disclosed herein, administered in combination with an anti-inflammatory active pharmaceutical ingredient, is about 25 mg, about 50 mg, about 75 mg, about 100 mg, about 125 mg, about 150 mg, about 175 mg, about 200 mg, about 225 mg, or about 250 mg. In some embodiments, an antiviral active pharmaceutical ingredient disclosed herein is administered either alone or administered in combination with an anti-inflammatory active pharmaceutical ingredient, in a dosing regimen of once a day, twice a day (b.i.d.), three times a day (t.i.d.), four times a day, or five times a day.

In some embodiments, an effective dosage per kg (of the subject mammal) of an antiviral active pharmaceutical ingredient disclosed herein, administered in combination with an anti-inflammatory active pharmaceutical ingredient, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an antiviral active pharmaceutical ingredient disclosed herein, administered in combination with an anti-inflammatory active pharmaceutical ingredient, is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an anti-inflammatory active pharmaceutical ingredient disclosed herein, including a steroid, administered in combination with an antiviral active pharmaceutical ingredient, is in the range of about 1 mg to about 300 mg, about 10 mg to about 250 mg, about 20 mg to about 225 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an anti-inflammatory active pharmaceutical ingredient disclosed herein, including a steroid, administered in combination with an antiviral active pharmaceutical ingredient, is about 25 mg, about 50 mg, about 75 mg, about 100 mg, about 125 mg, about 150 mg, about 175 mg, about 200 mg, about 225 mg, or about 250 mg. In some embodiments, an anti-inflammatory active pharmaceutical ingredient disclosed herein, including a steroid, is administered either alone or administered in combination with an antiviral active pharmaceutical ingredient, in a dosing regimen of once a day, twice a day (b.i.d.), three times a day (t.i.d.), four times a day, or five times a day.

In some embodiments, an effective dosage per kg (of the subject mammal) of an anti-inflammatory active pharmaceutical ingredient disclosed herein, administered in combination with an antiviral active pharmaceutical ingredient, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an anti-inflammatory active pharmaceutical ingredient disclosed herein, administered in combination with an antiviral active pharmaceutical ingredient, is about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a dosage of 10 to 200 mg b.i.d., including 50, 60, 70, 80, 90, 100 or 150 mg b.i.d., is provided for the antiviral active pharmaceutical ingredient, and a dosage of 10 to 300 mg b.i.d., including 25, 50, 75, 100, 150 or 200 mg b.i.d., is provided for the anti-inflammatory active pharmaceutical ingredient, including a steroid. In some instances, dosage levels below the lower limit of the aforesaid range may be more than adequate, while in other cases still larger doses may be employed without causing any harmful side effect—e.g., by dividing such larger doses into several small doses for administration throughout the day.

In selected embodiments, the combination of the anti-inflammatory active pharmaceutical ingredient and antiviral active pharmaceutical ingredients is administered in a single dose. In selected embodiments, such administration will be by injection—e.g., intravenous injection, in order to introduce the agents quickly. However, other routes may be used as appropriate. A single dose of the combination of the anti-inflammatory and antiviral active pharmaceutical ingredients may also be used for treatment of an acute condition.

In selected embodiments, the combination of the anti-inflammatory active pharmaceutical ingredient and antiviral active pharmaceutical ingredients is administered in multiple doses. Dosing may be once, twice, three times, four times, five times, six times, or more than six times per day. Dosing may be about once a month, once every two weeks, once a week, or once every other day. In other embodiments, the combination of the anti-inflammatory active pharmaceutical ingredient and antiviral active pharmaceutical ingredients is administered about once per day to about 6 times per day. In another embodiment the administration of the combination of the anti-inflammatory active pharmaceutical ingredient and antiviral active pharmaceutical ingredients continues for less than about 7 days. In yet another embodiment the administration continues for more than about 6, 10, 14, 28 days, two months, six months, or one year. In some cases, continuous dosing is achieved and maintained as long as necessary.

Administration of the active pharmaceutical ingredients of the invention may continue as long as necessary. In selected embodiments, the combination of the anti-inflammatory and antiviral active pharmaceutical ingredients is administered for more than 1, 2, 3, 4, 5, 6, 7, 14, or 28 days. In some embodiments, the combination of the anti-inflammatory and antiviral active pharmaceutical ingredients is administered for less than 28, 14, 7, 6, 5, 4, 3, 2, or 1 day. In selected embodiments, the combination of the anti-inflammatory and antiviral active pharmaceutical ingredients is administered chronically on an ongoing basis—e.g., for the treatment of chronic effects.

Administration of the active pharmaceutical ingredients of the invention may be tapered or decreased over a defined period. A kit may be provided to facilitate the tapered administration protocol. In an embodiment, the treatment is tapered over a period of 3 days, 4 days, 5 days, 6 days, 7 days, 8 days, 9 days, or 10 days. In an embodiment, the treatment is tapered over 7 days, with a constant dose administered 4 times a day on day 1, 3 times a day on day 2, 2 times a day on day 3, and once a day on days 4, 5, 6, and 7.

An effective amount of the combination of the antiviral active pharmaceutical ingredient and an anti-inflammatory active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

Methods of Treating Viral Infections with Combinations of Antiviral Active Pharmaceutical Ingredients and Anti-Inflammatory Active Pharmaceutical Ingredients, Including Steroids

In selected embodiments, the invention provides a method of treating or interpatient and intrapatient propylaxis of a viral infection in a mammal that comprises administering to said mammal a therapeutically effective amount of a antiviral active pharmaceutical ingredient and an anti-inflammatory active pharmaceutical ingredient, such as a steroid, or a pharmaceutically acceptable salt or ester, prodrug, solvate or hydrate of either or both of the antiviral active pharmaceutical ingredient or the anti-inflammatory active pharmaceutical ingredient. In an embodiment, the invention provides a method of treating or interpatient and intrapatient propylaxis of acute adenovirus conjunctivitis, also known as pinkeye. In an embodiment, the invention provides a method of treating or interpatient and intrapatient propylaxis of bulbar conjunctival redness. In an embodiment, the invention provides a method of treating or interpatient and intrapatient propylaxis of watery conjunctival discharge. In an embodiment, the invention provides a method of treating or interpatient and intrapatient propylaxis of vision loss from conjunctival infection sequela such as the formation of infiltrates.

In an embodiment, the viral infection is an infection of the eye. In an embodiment, the viral infection of the eye is adenoviral conjunctivus. In an embodiment, the viral infection of the eye is ocular conjunctival or corneal infection caused by a virus.

In an embodiment, the viral infection is selected from the group consisting of a viral infection of the eye, a viral infection of the salivary glands, a viral infection of the skin, a viral infection of the liver, a viral infection of the ear, a viral infection of the brain or spine, viral meningitis infection, a viral infection of the lung, a viral infection of the nasal mucosa, and a systematic viral infection, and a viral infection of a mucous membrane.

In an embodiment, the viral infection of the eye is selected from the group consisting of adenovirus infection, cytomegalovirus infection, herpes simplex virus infection, and varicella zoster infection, and viral conjunctivitis.

In an embodiment, the viral infection of the salivary glands is selected from the group consisting of cytomegalovirus infection, mumps infection, coxsackie virus infection, parainfluenza infection, influenza A infection, parvovirus B19 infection, and herpes virus infection.

In an embodiment, the viral infection of the skin is selected from the group consisting of varicella infection, herpes simplex infection, herpes zoster infection, and poxvirus infection.

In an embodiment, the viral infection of the liver is selected from the group consisting of hepatitis A infection, hepatitis B infection, hepatitis C infection, hepatitis D infection, hepatitis E infection, and hepatitis G infection.

In an embodiment, the viral infection of the brain or spine is selected from the group consisting of arbovirus infection, coxsackievirus infection, echovirus infection, and enterovirus infection.

In an embodiment, the viral menegitis infection is selected from the group consisting of enterovirus infection, echovirus infection, poliovirus infection, Coxsackie A virus infection, herpesviridae infection, herpes simplex virus type 1 infection, herpes simplex virus type 2 infection, varicella zoster infection, Epstein-Barr virus infection, cytomegalovirus infection, human immunodeficiency virus infection, La Crosse virus infection, lymphocytic choriomeningitis virus infection, measles virus infection, mumps virus infection, St. Louis encephalitis virus infection, and West Nile virus infection.

In an embodiment, the viral infection of the ear is selected from the group consisting of mononucleosis infection, herpes virus infection, influenza infection, rubella infection, mumps infection, poliovirus infection, hepatitis virus poliovirus, Epstein-Barr virus infection, and measles infection.

In an embodiment, the viral infection of the nasal mucosa is selected from the group consisting of rhinovirus infection, rotavirus infection, alpha coronavirus infection, beta coronavirus infection, gamma coronavirus infection, delta coronavirus infection, influenza virus infection, parainfluenza virus infection, respiratory syncytial virus infection, enterovirus infection, and metapneumovirus infection.

In an embodiment, the systemic viral infection is selected from the group consisting of human immunodeficiency virus 1 infection, of human immunodeficiency virus 1 infection, influenza virus infection, rotavirus infection, mononucleosis infection, and measles infection.

In an embodiment, the antiviral active pharmaceutical ingredient used in the foregoing methods is selected from the group consisting of povidone-iodine (PVP-I), abacavir, aciclovir, adefovir, adefovir dipivoxil, amantadine, amprenavir, atazanavir, boceprevir, cidofovir, cobicistat, dolutegravir, darunavir, delavirdine, didanosine, docosanol, edoxudine, efavirenz, elvitegravir, emtricitabine, enfuvirtide, entecavir, famciclovir, fomivirsen, fosamprenavir, foscarnet, ganciclovir, ibacitabine, idoxuridine, imiquimod, indinavir, lamivudine, lopinavir, loviride, maraviroc, moroxydine, methisazone, nelfinavir, nevirapine, oseltamivir, penciclovir, peramivir, pleconaril, podophyllotoxin, raltegravir, ribavirin, rilpivirine, rimantadine, ritonavir, saquinavir, sofosbuvir, stavudine, telaprevir, tenofovir, tenofovir disoproxil, tipranavir, trifluridine, tromantadine, valaciclovir, valganciclovir, vicriviroc, vidarabine, viramidine, zalcitabine, zanamivir, and zidovudine, or a pharmaceutically-accepted salt or ester thereof.

In an embodiment, the anti-inflammatory active pharmaceutical ingredient used in the foregoing methods is a steroid selected from the group consisting of dexamethasone, dexamethasone alcohol, dexamethasone sodium phosphate, fluromethalone acetate, fluormethalone acetate, fluromethalone alcohol, lotoprednol etabonate, medrysone, prednisolone acetate, prednisolone sodium phosphate, difluprednate, rimexolone, hydrocortisone, hydrocortisone acetate, lodoxamide tromethamine, fluticasone, fluticasone propionate, fluticasone furoate, or a pharmaceutically-acceptable salt or ester thereof.

In an embodiment, the anti-inflammatory active pharmaceutical ingredient used in the foregoing methods is a non-steroidal anti-inflammatory drug (NSAID) selected from the group consisting of bromfenac, nepafenac, diclofenac, ketorolac, ketorlac tromethamine, flurbiprofen, suprofen, celecoxib, naproxen, rofecoxib, salts, derivatives, prodrugs, and esters thereof, and any combinations thereof.

In an embodiment, the viral infection is an infection of the eye. In an embodiment, the viral infection is an infection of the eyelid, such as blepharitis (eyelid inflammation).

In an embodiment, the invention provides a method of treating a viral infection using a combination of ganciclovir and a steroid. In an embodiment, the invention provides a method of treating a viral infection using a combination of sofosbuvir and a steroid.

In an embodiment, the invention provides a method of treating a viral infection using a combination of fluticasone, or a salt or ester thereof, and PVP-I. In an embodiment, the combination of fluticasone or a salt or ester thereof and PVP-I may be administered topically.

### Other Methods and Compositions

In an embodiment, the antiviral active pharmaceutical ingredient is replaced by cyclosporin. In an embodiment, the invention provides a pharmaceutical composition comprising cyclosporin and PVP-I. In an embodiment, the invention provides a method of treating an infection using a composition comprising cyclosporin and PVP-I.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—Synergistic Combination of Dexamethasone and PVP-I

A randomized, double masked, multi-center study was conducted to investigate the safety and efficacy of the combination dexamethasone and PVP-I therapy for the treatment of acute adenoviral conjunctivitis. The dexamethasone/PVP-I combination therapy functions according to a dual mechanism, with the dexamethasone component serving to reduce inflammation while the PVP-I component serving to eliminate the virus. Results in this novel study demonstrate that this combination treatment was safe and well-tolerated by patients with no serious adverse events, in addition to data showing clinical efficacy and viral eradication. There is a significant unmet medical need for this treatment as currently, no therapy is available.

Selection of participants in the trial was conducted under the following guidelines. Enrollment was based on patients who reported signs of viral conjunctivitis for up to 5 days prior to the first visit of the study in at least one eye. Signs of viral conjunctivitis were quantitatively evaluated by measuring the patient's bulbar conjunctival redness on a 0-3 scale (minimum grade of 1 needed for enrollment) and watery conjunctival discharge on a 0-3 scale (minimum grade of 1 needed for enrollment). Prospective enrollees were also required to have tested positive for viral conjunctivitis as diagnosed using readily available, in-office tests. For example, adenoviral antigens may be detected directly from human eye fluid along the inside lining of the lower eyelid (the palpebral conjunctiva). Finally, participants were required to have positive adenoviral cell culture for analysis using cell culture with confirmatory immunofluorescence testing.

Patients were selected based on clinical signs of acute viral conjunctivitis (e.g. bulber conjunctival redness and watery conjunctival discharge) and tested positive for an adenoviral antigen by the RPS ADENOPLUS test. In total, 176 subjects were tested, with 59 randomly being chosen to test the dexamethasone and PVP-I combination therapy, 59 randomly chosen to test solely PVP-I, and 58 randomly chosen for the placebo group (dexamethasone/PVP-I vehicle). Of the subjects selected, the pacebo group suffered the highest discontinuation rate (24 individuals or 41.4% as compared to 12 individuals for the dexamethasone/PVP-I group-20.7%—and 15 individuals for the PVP-I group—25.4%). All demographic characteristics were well-balanced across treatment groups.

Under the treatment and evaluation schedule, patients administered one drop in both eyes four times a day for five days. Patients were evaluated after completion of the treatment course on 1, 3, 6, and 12 days after administration. Scoring on the patient's response to their treatment was performed by a trained ophthalmologist who compared the patient's eye exam to the Ora redness scale, a standardized picture scale and an assessment of watery conjunctival discharge following Ora's written definitions. Signs were scored on a scale of zero to three using Ora's CALIBRA scales.

Results comparing the dexamethasone/PVP-I combination therapy, PVP-I treatment, and the placebo are summarized in Table 2 below and show that the dexamethasone/PVP-I combination nearly completely resolves the signs and symptoms of adenoviral conjunctivitis. For example, dexamethasone/PVP-I was statistically better than both vehicle and PVP-I for discharge alone at Visit 3 (p-value equals 0.0266, data not shown). Furthermore, there was a trend towards statistical significance between the dexamethasone/PVP-I and PVP-I for both redness and watery conjunctival discharge at Visit 3.

TABLE 2
Results comparing the dexamethasone/PVP-I combination
therapy, PVP-I treatment, and the placebo.

| | | |
|---|---|---|
| Dexamethasone | | |
| (0.1%)/ | | Dexamethasone/ |
| PVP-I (0.6%) | 0.6% PVP-I | PVP-I vehicle |

Primary Study Eye, mITT, LOCF, (Endpoint:
Redness and Discharge to ZERO)

| | | | |
|---|---|---|---|
| Visit 2 | | | |
| Clinical Cure | 0% | 0% | 0% |
| Visit 3 | | | |
| Clinical Cure | 15 (31.3%) | 9 (18.0%) | 5 (10.9%) |
| p-value v. | 0.0158 | 0.3227 | — |
| Dexamethasone/ | | | |
| PVP-I vehicle | | | |
| p-value v. PVP-I | 0.1273* | — | — |
| Visit 4 | | | |
| Clinical Cure | 30 (62.5%) | 31 (62.0%) | 21 (45.7%) |
| p-value v. | 0.1012 | 0.1083 | — |
| Dexamethasone/ | | | |
| PVP-I vehicle | | | |
| p-value v. PVP-I | 0.9593 | — | — |

*p-value becomes 0.0461 if one patient in the PVP-I arm is switched to the dexamethasone/PVP-I arm.

Results from Table 3 below show that the dexamethasone/PVP-I combination had substantial clinical benefit using the expanded clinical cure definition. For example, dexamethasone/PVP-I was statistically better than vehicle at all visits and better than PVP-I at visits 2 and 3, with a strong trend at visit 4. There was no statistical significance between PVP-I and vehicle.

TABLE 3
Clinical benefit.

| | Dexamethasone | | |
|---|---|---|---|
| Redness and | (0.1%)/PVP-I | | Dexamethasone/ |
| Discharge ≤1 | (0.6%) | 0.6% PVP-I | PVP-I vehicle |

Primary Study Eye, mITT, LOCF, (Endpoint:
Redness and Discharge; 0-0, 0-1, 1-0)

| | | | |
|---|---|---|---|
| Visit 2 | | | |
| Expanded Clinical | 17 (35.4%) | 9 (18.0%) | 4 (8.7%) |
| Cure | | | |
| p-value v. | 0.0025 | 0.2382 | — |
| Dexamethasone/PVP- | | | |
| I vehicle | | | |
| p-value v. PVP-I | 0.0509 | — | — |
| Visit 3 | | | |
| Expanded Clinical | 37 (77.1%) | 26 (52.0%) | 20 (43.5%) |
| Cure | | | |
| p-value v. | 0.0009 | 0.4037 | — |
| Dexamethasone/PVP- | | | |
| I vehicle | | | |
| p-value v. PVP-I | 0.0096 | — | — |
| Visit 4 | | | |
| Expanded Clinical | 46 (95.8%) | 42 (84.0%) | 33 (71.7%) |
| Cure | | | |
| p-value v. | 0.0016 | 0.1466 | — |
| Dexamethasone/PVP- | | | |
| I vehicle | | | |
| p-value v. PVP-I | 0.0920 | — | — |

Results from Table 4 below show that the dexamethasone/PVP-I combination significantly eradicates adenovirus, i.e. the dexamethasone/PVP-I combination demonstrates potential synergy in adenoviral eradication. For example, dexamethasone/PVP-I was statistically better than vehicle at all visits and there was a strong trend towards statistical significance between dexamethasone/PVP-I and PVP-I at visit 3. Furthermore, PVP-I was statistically better than vehicle at visit 2.

TABLE 4
Eradication of adenovirus.

| | | |
|---|---|---|
| Dexamethasone | | |
| (0.1%)/ | | Dexamethasone/ |
| PVP-I (0.6%) | 0.6% PVP-I | PVP-I vehicle |

Primary Study Eye, mITT, LOCF, (Endpoint:
Adenovirus Cell Culture Negative)

| | | | |
|---|---|---|---|
| Visit 2 | | | |
| Adenoviral Eradication | 17 (35.4%) | 16 (32.0%) | 4 (8.7%) |
| p-value v. | 0.0025 | 0.0057 | — |
| Dexamethasone/ | | | |
| PVP-I vehicle | | | |
| p-value v. PVP-I | 0.7205 | — | — |
| Visit 3 | | | |
| Adenoviral Eradication | 38 (79.2%) | 31 (62.0%) | 26 (56.5%) |
| p-value v. | 0.0186 | 0.5851 | — |
| Dexamethasone/ | | | |
| PVP-I vehicle | | | |
| p-value v. PVP-I | 0.0627* | — | — |
| Visit 4 | | | |
| Adenoviral Eradication | 46 (95.8%) | 42 (84.0%) | 35 (76.1%) |
| p-value v. | 0.0068 | 0.3310 | — |
| Dexamethasone/ | | | |
| PVP-I vehicle | | | |
| p-value v. PVP-I | 0.0920 | — | — |

*p-value becomes 0.0212 if one patient in the PVP-I arm is switched to the dexamethasone/PVP-I arm.

Results from Table 5 below show that the dexamethasone/PVP-I combination has significant potential as a clinical cure and to significantly eradicate adenovirus. For example, dexamethasone/PVP-I was statistically better than vehicle at visits 3 and 4 and there was a trend towards a statistical significance between dexamethasone/PVP-I and PVP-I at visit 3.

TABLE 5
Clinical cure results.

| | | |
|---|---|---|
| Dexamethasone | | |
| (0.1%)/ | | Dexamethasone/ |
| PVP-I (0.6%) | 0.6% PVP-I | PVP-I vehicle |

Primary Study Eye, mITT, LOCF, (Endpoint:
Clinical Cure + Adenovirus Cell Culture Negative)

| | | | |
|---|---|---|---|
| Visit 2 | | | |
| Clinical Cure + | 0% | 0% | 0% |
| Adenoviral | | | |
| Eradication | | | |
| Visit 3 | | | |
| Clinical Cure + | 15 (30.6%) | 9 (18.0%) | 3 (6.4%) |
| Adenoviral | | | |
| Eradication | | | |
| p-value v. | 0.0033 | 0.1229 | — |
| Dexamethasone/ | | | |
| PVP-I vehicle | | | |
| p-value v. PVP-I | 0.1432* | — | — |
| Visit 4 | | | |
| Clinical Cure + | 29 (60.4%) | 29 (58.0%) | 19 (39.6%) |
| Adenoviral | | | |
| Eradication | | | |
| p-value v. | 0.0412 | 0.0683 | — |
| Dexamethasone/ | | | |
| PVP-I vehicle | | | |
| p-value v. PVP-I | 0.8078 | — | — |

*p-value becomes 0.0532 if one patient in the PVP-I arm is switched to the dexamethasone/PVP-I arm.

Adverse effects were similar between the dexamethasone/PVP-I combination and the dexamethasone/PVP-I vehicle (placebo group). Furthermore, no adverse events were related to treatment. Ocular treatment emergent adverse effects (TEAEs) rates were not statistically significant between the treatments. Table 6 below illustrates the most common ocular TEAEs observed during the study and their rates.

TABLE 6
Observed ocular treatment emergent adverse effects (TEAEs).

| | | Observed ocular treatment emergent | | |
|---|---|---|---|---|
| | | adverse effects (TEAEs) | | |
| | | Corneal | Punctate | Eyelid |
| | | Infiltrates | Keratitis | Edema |
| Rates | Dexamethasone/PVP-I | 11 (19.0%) | 13 (22.4%) | 7 (12.1%) |
| | PVP-I | 18 (30.5%) | 16 (27.1%) | 16 (27.1%) |
| | Dexamethasone/PVP-I | 12 (20.7%) | 11 (19.0%) | 12 (20.7%) |
| | vehicle | | | |

Therefore, results in this novel study demonstrate that this combination treatment was safe and well-tolerated by patients with no serious adverse events, in addition to data showing clinical efficacy and viral eradication. Specifically, the dexamethasone/PVP-I combination effectively treats acute adenoviral conjunctivitis and provides clinically meaningful benefit and rapid viral eradication. Broad clinically meaningful efficacy was achieved based on clinical cure, viral eradication, clinical cure and viral eradication, expanded clinical cure, and potential synergistic efficacy (i.e. dexamethasone added to PVP-I improves viral eradication). It is contemplated that the dexamethasone/PVP-I combination may be used towards the treatment of any red eye conditions, blepharitis (anterior (bacterial) and posterior (inflammatory)), infectious conjunctivitis, allergic conjunctivitis, foreign bodies, and in pre-op/post-op surgery or intraocular injections.

Fellow eye effects (i.e. intrapatient infection) were also evaluated in a separate clinical study. In this study, the number of patients with cross infection to the fellow eye (defined as any increase in the total clinical score in the fellow eye) on day 6 was 13 (20% of the arm) with placebo compared to only 5 (8% of the arm) using dexamethasone/PVP-I.

The combined synergy of dexamethasone/PVP-I treatment methods leads to unexpectedly superior (1) viral eradation and (2) clinical cure over PVP-I alone.

### Example 2—Rabbit Study of Dexamethasone and PVP-I

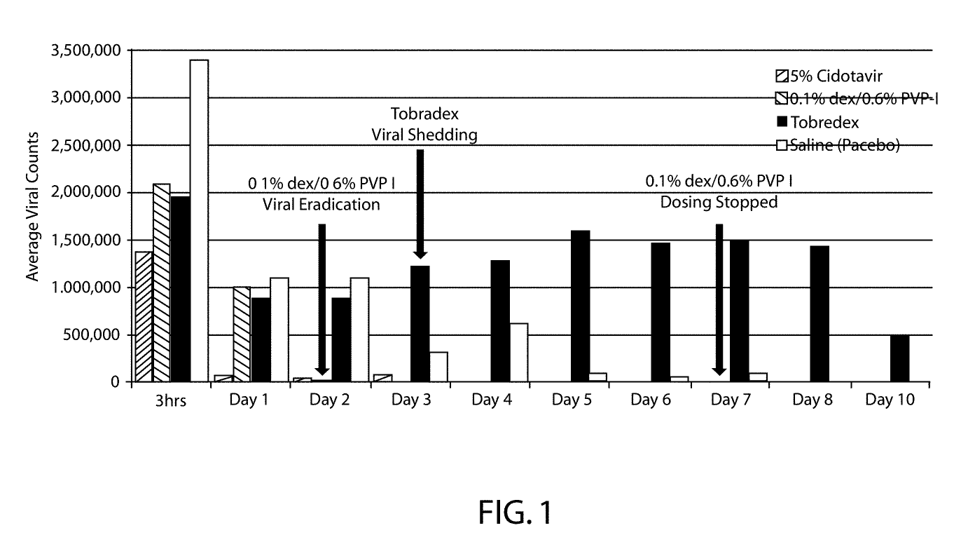

A rabbit study of a dexamethasone/PVP-I formulation has also been reported, as described in Clement, et al., Invest. Ophthalmol. & Visual Sci., 2011, 52, 339-344. Key results of this study are illustrated in FIG. 1. The results illustrate rapid and statistically significant clinical improvement over both placebo and active controls. The results of the human study described above are synergistic and are unexpectedly superior to the results obtained in the rabbit model.

1.-22. (canceled)

23. A method of treating acute adenoviral conjunctivitis in a subject in need thereof, comprising administering to the subject a composition comprising:
povidone-iodine (PVP-I) at a concentration of about 0.6%; and
dexamethasone at a concentration of about 0.1%,
wherein administration of the composition to a population of subjects provides significant improvement in the signs and symptoms of adenoviral conjunctivitis over a period of at least about 1 to about 12 days after administration as measured by a validated rating scale.

24. The method of claim 23, wherein the treating nearly completely resolves the signs and symptoms of adenoviral conjunctivitis.

25. The method of claim 23, wherein the treating results in viral eradication.

26. The method of claim 23, wherein the improvement in the signs and symptoms of adenoviral conjunctivitis is measured using the Oral redness CALIBRA scale.

27. The method of claim 23, wherein the improvement in the signs and symptoms of adenoviral conjunctivitis is measured using the expanded clinical cure definition.

28. The method of claim 23, wherein the treating is well-tolerated by the subject with no serious adverse events.

29. The method of claim 23, wherein the composition is an eye drop solution.

30. The method of claim 23, wherein the administering is four times a day administration.

31. The method of claim 23, comprising administering one drop of the composition in both eyes four times a day.

32. The method of claim 23, wherein the subject is a human subject.
